SEQUENCE LISTING (95 sequences)

SEQ ID NO: 1 - protein, 10 aa, Homo sapiens
ALWGPDPAAA

SEQ ID NO: 2 - protein, 205 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLCAMRGDSSYKLIFGSGTRLLVRPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 3 - protein, 246 aa, Homo sapiens
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSLWEKLAKNIQYFGAGTRLSVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 4 - protein, 205 aa, artificial sequence
  TCR alpha chain extracellular region
QKEVEQDPGPLSVPEGAIVSLNCTYSNSAFQYFMWYRQYSRKGPELLMYTYSSGNKEDGRFTAQVDKSSKYISLFIRDSQPSDSATYLCAMRGDSSYKLIFGSGTRLLVRPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 5 - protein, 246 aa, artificial sequence
  TCR beta chain extracellular region
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSLWEKLAKNIQYFGAGTRLSVLEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 6 - DNA, 618 bp, artificial sequence
  TCR alpha chain extracellular region
atgcaaaaagaagttgaacaagatcctggaccactcagtgttccagagggagccattgtttctctcaactgcacttacagcaacagtgcttttcaatacttcatgtggtacagacagtattccagaaaaggccctgagttgctgatgtacacatactccagtggtaacaaagaagatggaaggtttacagcacaggtcgataaatccagcaagtatatctccttgttcatcagagactcacagcccagtgattcagccacctacctctgtgcaatgaggggggatagcagctataaattgatcttcgggagtgggaccagactgctggtcaggcctgatatccagaaccctgaccctgccgtgtaccagctgagagactctaagtcgagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcc

SEQ ID NO: 7 - DNA, 744 bp, artificial sequence
  TCR beta chain extracellular sequence
atggatgctggagttattcaatcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtttatgggagaagttagccaaaaacattcagtacttcggcgccgggacccggctctcagtgctggaggacctgaaaaacgtgttcccacccgaggtcgctgtgtttgagccatcagaagcagagatctcccacacccaaaaggccacactggtgtgcctggccaccggtttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagactaa

SEQ ID NO: 8 - protein, 112 aa, artificial sequence
  TCR alpha chain variable region
QKEVEQDPGPLSVPEGAIVSLNCTYSNQYATRFMWYRQYSRKGPELLMYTYSSGNKEDGRFTAQVDKSSKYISLFIRDSQPSDSATYLCAMRGDSSYKLIFGSGTRLLVRPD

SEQ ID NO: 9 - protein, 112 aa, artificial sequence
  TCR alpha chain variable region
QKEVEQDPGPLSVPEGAIVSLNCTYSNPYMQAFMWYRQYSRKGPELLMYTYSSGNKEDGRFTAQVDKSSKYISLFIRDSQPSDSATYLCAMRGDSSYKLIFGSGTRLLVRPD

SEQ ID NO: 10 - protein, 112 aa, artificial sequence
  TCR alpha chain variable region
QKEVEQDPGPLSVPEGAIVSLNCTYSNDFMSRFMWYRQYSRKGPELLMYTYSSGNKEDGRFTAQVDKSSKYISLFIRDSQPSDSATYLCAMRGDSSYKLIFGSGTRLLVRPD

SEQ ID NO: 11 - protein, 112 aa, artificial sequence
  TCR alpha chain variable region
QKEVEQDPGPLSVPEGAIVSLNCTYSNYQYKVFMWYRQYSRKGPELLMYTYSSGNKEDGRFTAQVDKSSKYISLFIRDSQPSDSATYLCAMRGDSSYKLIFGSGTRLLVRPD

SEQ ID NO: 12 - protein, 112 aa, artificial sequence
  TCR alpha chain variable region
QKEVEQDPGPLSVPEGAIVSLNCTYSNQLMTAFMWYRQYSRKGPELLMYTYSSGNKEDGRFTAQVDKSSKYISLFIRDSQPSDSATYLCAMRGDSSYKLIFGSGTRLLVRPD

SEQ ID NO: 13 - protein, 112 aa, artificial sequence
  TCR alpha chain variable region
QKEVEQDPGPLSVPEGAIVSLNCTYSNTQFTRFMWYRQYSRKGPELLMYTYSSGNKEDGRFTAQVDKSSKYISLFIRDSQPSDSATYLCAMRGDSSYKLIFGSGTRLLVRPD

SEQ ID NO: 14 - protein, 112 aa, artificial sequence
  TCR alpha chain variable region
QKEVEQDPGPLSVPEGAIVSLNCTYSNSAFQYFMWYRQYSRKGPELLMYCQRVCNKEDGRFTAQVDKSSKYISLFIRDSQPSDSATYLCAMRGDSSYKLIFGSGTRLLVRPD

SEQ ID NO: 15 - protein, 112 aa, artificial sequence
  TCR alpha chain variable region
QKEVEQDPGPLSVPEGAIVSLNCTYSNSAFQYFMWYRQYSRKGPELLMYGSLRHNKEDGRFTAQVDKSSKYISLFIRDSQPSDSATYLCAMRGDSSYKLIFGSGTRLLVRPD

SEQ ID NO: 16 - protein, 112 aa, artificial sequence
  TCR alpha chain variable region
QKEVEQDPGPLSVPEGAIVSLNCTYSNQYATRFMWYRQYSRKGPELLMYGSLRHNKEDGRFTAQVDKSSKYISLFIRDSQPSDSATYLCAMRGDSSYKLIFGSGTRLLVRPD

SEQ ID NO: 17 - protein, 112 aa, artificial sequence
  TCR alpha chain variable region
QKEVEQDPGPLSVPEGAIVSLNCTYSNDFMSRFMWYRQYSRKGPELLMYGSLRHNKEDGRFTAQVDKSSKYISLFIRDSQPSDSATYLCAMRGDSSYKLIFGSGTRLLVRPD

SEQ ID NO: 18 - protein, 112 aa, artificial sequence
  TCR alpha chain variable region
QKEVEQDPGPLSVPEGAIVSLNCTYSNTQFTRFMWYRQYSRKGPELLMYGSLRHNKEDGRFTAQVDKSSKYISLFIRDSQPSDSATYLCAMRGDSSYKLIFGSGTRLLVRPD

SEQ ID NO: 19 - protein, 114 aa, artificial sequence
  TCR alpha chain variable region
QKEVEQDPGPLSVPEGAIVSLNCTYSNQYATRFMWYRQYSRKGPELLMYGGSLRHHNKEDGRFTAQVDKSSKYISLFIRDSQPSDSATYLCAMRGDSSYKLIFGSGTRLLVRPD

SEQ ID NO: 20 - protein, 114 aa, artificial sequence
  TCR alpha chain variable region
QKEVEQDPGPLSVPEGAIVSLNCTYSNQYATRFMWYRQYSRKGPELLMYGGSLRHNKEDDGRFTAQVDKSSKYISLFIRDSQPSDSATYLCAMRGDSSYKLIFGSGTRLLVRPD

SEQ ID NO: 21 - protein, 114 aa, artificial sequence
  TCR alpha chain variable region
QKEVEQDPGPLSVPEGAIVSLNCTYSNQYATRFMWYRQYSRKGPELLMYGGSLRHGNKEDGRFTAQVDKSSKYISLFIRDSQPSDSATYLCAMRGDSSYKLIFGSGTRLLVRPD

SEQ ID NO: 22 - protein, 114 aa, artificial sequence
  TCR alpha chain variable region
QKEVEQDPGPLSVPEGAIVSLNCTYSNSALRPQLFMWYRQYSRKGPELLMYTYSSGNKEDGRFTAQVDKSSKYISLFIRDSQPSDSATYLCAMRGDSSYKLIFGSGTRLLVRPD

SEQ ID NO: 23 - protein, 114 aa, artificial sequence
  TCR alpha chain variable region
QKEVEQDPGPLSVPEGAIVSLNCTYSNSAMQPVLFMWYRQYSRKGPELLMYTYSSGNKEDGRFTAQVDKSSKYISLFIRDSQPSDSATYLCAMRGDSSYKLIFGSGTRLLVRPD

SEQ ID NO: 24 - protein, 114 aa, artificial sequence
  TCR alpha chain variable region
QKEVEQDPGPLSVPEGAIVSLNCTYSNSAYWPTQFMWYRQYSRKGPELLMYTYSSGNKEDGRFTAQVDKSSKYISLFIRDSQPSDSATYLCAMRGDSSYKLIFGSGTRLLVRPD

SEQ ID NO: 25 - protein, 115 aa, artificial sequence
  TCR alpha chain variable region
QKEVEQDPGPLSVPEGAIVSLNCTYSNSAFQYPHMFMWYRQYSRKGPELLMYTYSSGNKEDGRFTAQVDKSSKYISLFIRDSQPSDSATYLCAMRGDSSYKLIFGSGTRLLVRPD

SEQ ID NO: 26 - protein, 115 aa, artificial sequence
  TCR alpha chain variable region
QKEVEQDPGPLSVPEGAIVSLNCTYSNSAFNKYTAFMWYRQYSRKGPELLMYTYSSGNKEDGRFTAQVDKSSKYISLFIRDSQPSDSATYLCAMRGDSSYKLIFGSGTRLLVRPD

SEQ ID NO: 27 - protein, 115 aa, artificial sequence
  TCR alpha chain variable region
QKEVEQDPGPLSVPEGAIVSLNCTYSNSAFNWPQVFMWYRQYSRKGPELLMYTYSSGNKEDGRFTAQVDKSSKYISLFIRDSQPSDSATYLCAMRGDSSYKLIFGSGTRLLVRPD

SEQ ID NO: 28 - protein, 115 aa, artificial sequence
  TCR alpha chain variable region
QKEVEQDPGPLSVPEGAIVSLNCTYSNSGYDQPQYFMWYRQYSRKGPELLMYTYSSGNKEDGRFTAQVDKSSKYISLFIRDSQPSDSATYLCAMRGDSSYKLIFGSGTRLLVRPD

SEQ ID NO: 29 - protein, 115 aa, artificial sequence
  TCR alpha chain variable region
QKEVEQDPGPLSVPEGAIVSLNCTYSNSAFLWPAQFMWYRQYSRKGPELLMYTYSSGNKEDGRFTAQVDKSSKYISLFIRDSQPSDSATYLCAMRGDSSYKLIFGSGTRLLVRPD

SEQ ID NO: 30 - protein, 115 aa, artificial sequence
  TCR alpha chain variable region
QKEVEQDPGPLSVPEGAIVSLNCTYSNSAFQYPTMFMWYRQYSRKGPELLMYTYSSGNKEDGRFTAQVDKSSKYISLFIRDSQPSDSATYLCAMRGDSSYKLIFGSGTRLLVRPD

SEQ ID NO: 31 - protein, 115 aa, artificial sequence
  TCR alpha chain variable region
QKEVEQDPGPLSVPEGAIVSLNCTYSNSAFQQFTRFMWYRQYSRKGPELLMYTYSSGNKEDGRFTAQVDKSSKYISLFIRDSQPSDSATYLCAMRGDSSYKLIFGSGTRLLVRPD

SEQ ID NO: 32 - protein, 115 aa, artificial sequence
  TCR alpha chain variable region
QKEVEQDPGPLSVPEGAIVSLNCTYSERQYDAFQYFMWYRQYSRKGPELLMYTYSSGNKEDGRFTAQVDKSSKYISLFIRDSQPSDSATYLCAMRGDSSYKLIFGSGTRLLVRPD

SEQ ID NO: 33 - protein, 115 aa, artificial sequence
  TCR alpha chain variable region
QKEVEQDPGPLSVPEGAIVSLNCTYSNSQIKNPQYFMWYRQYSRKGPELLMYTYSSGNKEDGRFTAQVDKSSKYISLFIRDSQPSDSATYLCAMRGDSSYKLIFGSGTRLLVRPD

SEQ ID NO: 34 - protein, 115 aa, artificial sequence
  TCR alpha chain variable region
QKEVEQDPGPLSVPEGAIVSLNCTYSNSAFGQPQMFMWYRQYSRKGPELLMYTYSSGNKEDGRFTAQVDKSSKYISLFIRDSQPSDSATYLCAMRGDSSYKLIFGSGTRLLVRPD

SEQ ID NO: 35 - protein, 115 aa, artificial sequence
  TCR alpha chain variable region
QKEVEQDPGPLSVPEGAIVSLNCTYSNSAFQPNPMFMWYRQYSRKGPELLMYTYSSGNKEDGRFTAQVDKSSKYISLFIRDSQPSDSATYLCAMRGDSSYKLIFGSGTRLLVRPD

SEQ ID NO: 36 - protein, 114 aa, artificial sequence
  TCR alpha chain variable region
QKEVEQDPGPLSVPEGAIVSLNCTYSNSLYNQPQYMWYRQYSRKGPELLMYTYSSGNKEDGRFTAQVDKSSKYISLFIRDSQPSDSATYLCAMRGDSSYKLIFGSGTRLLVRPD

SEQ ID NO: 37 - protein, 114 aa, artificial sequence
  TCR alpha chain variable region
QKEVEQDPGPLSVPEGAIVSLNCTYSNSAFQYFMWYRQYSRKGPELLMYIPTFKSGNKEDGRFTAQVDKSSKYISLFIRDSQPSDSATYLCAMRGDSSYKLIFGSGTRLLVRPD

SEQ ID NO: 38 - protein, 114 aa, artificial sequence
  TCR alpha chain variable region
QKEVEQDPGPLSVPEGAIVSLNCTYSNSAFQYFMWYRQYSRKGPELLMYQSAFQSGNKEDGRFTAQVDKSSKYISLFIRDSQPSDSATYLCAMRGDSSYKLIFGSGTRLLVRPD

SEQ ID NO: 39 - protein, 115 aa, artificial sequence
  TCR alpha chain variable region
QKEVEQDPGPLSVPEGAIVSLNCTYSNSAFQYFMWYRQYSRKGPELLMYTYSNQQVSNKEDGRFTAQVDKSSKYISLFIRDSQPSDSATYLCAMRGDSSYKLIFGSGTRLLVRPD

SEQ ID NO: 40 - protein, 114 aa, artificial sequence
  TCR alpha chain variable region
QKEVEQDPGPLSVPEGAIVSLNCTYSNSAFQYFMWYRQYSRKGPELLMYVPGHASGNKEDGRFTAQVDKSSKYISLFIRDSQPSDSATYLCAMRGDSSYKLIFGSGTRLLVRPD

SEQ ID NO: 41 - protein, 115 aa, artificial sequence
  TCR alpha chain variable region
QKEVEQDPGPLSVPEGAIVSLNCTYSNSAFQYFMWYRQYSRKGPELLMYTYPQLDTGNKEDGRFTAQVDKSSKYISLFIRDSQPSDSATYLCAMRGDSSYKLIFGSGTRLLVRPD

SEQ ID NO: 42 - protein, 115 aa, artificial sequence
  TCR alpha chain variable region
QKEVEQDPGPLSVPEGAIVSLNCTYSNSAFQYFMWYRQYSRKGPELLMYQDMRISSGNKEDGRFTAQVDKSSKYISLFIRDSQPSDSATYLCAMRGDSSYKLIFGSGTRLLVRPD

SEQ ID NO: 43 - protein, 115 aa, artificial sequence
  TCR alpha chain variable region
QKEVEQDPGPLSVPEGAIVSLNCTYSNSAFQYFMWYRQYSRKGPELLMYTYVPRNKGNKEDGRFTAQVDKSSKYISLFIRDSQPSDSATYLCAMRGDSSYKLIFGSGTRLLVRPD

SEQ ID NO: 44 - protein, 115 aa, artificial sequence
  TCR alpha chain variable region
QKEVEQDPGPLSVPEGAIVSLNCTYSNSAFQYFMWYRQYSRKGPELLMYMPYHQSSGNKEDGRFTAQVDKSSKYISLFIRDSQPSDSATYLCAMRGDSSYKLIFGSGTRLLVRPD

SEQ ID NO: 45 - protein, 115 aa, artificial sequence
  TCR alpha chain variable region
QKEVEQDPGPLSVPEGAIVSLNCTYSNSAFQYFMWYRQYSRKGPELLMYQSTQLSSGNKEDGRFTAQVDKSSKYISLFIRDSQPSDSATYLCAMRGDSSYKLIFGSGTRLLVRPD

SEQ ID NO: 46 - protein, 115 aa, artificial sequence
  TCR alpha chain variable region
QKEVEQDPGPLSVPEGAIVSLNCTYSNSAFQYFMWYRQYSRKGPELLMYTYSNSTRYNKEDGRFTAQVDKSSKYISLFIRDSQPSDSATYLCAMRGDSSYKLIFGSGTRLLVRPD

SEQ ID NO: 47 - protein, 115 aa, artificial sequence
  TCR alpha chain variable region
QKEVEQDPGPLSVPEGAIVSLNCTYSNSAFQYFMWYRQYSRKGPELLMYTYMGSFYGNKEDGRFTAQVDKSSKYISLFIRDSQPSDSATYLCAMRGDSSYKLIFGSGTRLLVRPD

SEQ ID NO: 48 - protein, 115 aa, artificial sequence
  TCR alpha chain variable region
QKEVEQDPGPLSVPEGAIVSLNCTYSNSAFQYFMWYRQYSRKGPELLMYLPQPWSSGNKEDGRFTAQVDKSSKYISLFIRDSQPSDSATYLCAMRGDSSYKLIFGSGTRLLVRPD

SEQ ID NO: 49 - protein, 115 aa, artificial sequence
  TCR alpha chain variable region
QKEVEQDPGPLSVPEGAIVSLNCTYSNSAFQYFMWYRQYSRKGPELLMYTYSLLSLDNKEDGRFTAQVDKSSKYISLFIRDSQPSDSATYLCAMRGDSSYKLIFGSGTRLLVRPD

SEQ ID NO: 50 - protein, 115 aa, artificial sequence
  TCR alpha chain variable region
QKEVEQDPGPLSVPEGAIVSLNCTYSNSAFQYFMWYRQYSRKGPELLMYVQAITSSGNKEDGRFTAQVDKSSKYISLFIRDSQPSDSATYLCAMRGDSSYKLIFGSGTRLLVRPD

SEQ ID NO: 51 - protein, 118 aa, artificial sequence
  TCR alpha chain variable region
QKEVEQDPGPLSVPEGAIVSLNCTYSNSAFNWPQVFMWYRQYSRKGPELLMYQDMRISSGNKEDGRFTAQVDKSSKYISLFIRDSQPSDSATYLCAMRGDSSYKLIFGSGTRLLVRPD

SEQ ID NO: 52 - protein, 118 aa, artificial sequence
  TCR alpha chain variable region
QKEVEQDPGPLSVPEGAIVSLNCTYSNSAFNWPQVFMWYRQYSRKGPELLMYTYMGSFYGNKEDGRFTAQVDKSSKYISLFIRDSQPSDSATYLCAMRGDSSYKLIFGSGTRLLVRPD

SEQ ID NO: 53 - protein, 118 aa, artificial sequence
  TCR alpha chain variable region
QKEVEQDPGPLSVPEGAIVSLNCTYSNSAFNWPQVFMWYRQYSRKGPELLMYLPQPWSSGNKEDGRFTAQVDKSSKYISLFIRDSQPSDSATYLCAMRGDSSYKLIFGSGTRLLVRPD

SEQ ID NO: 54 - protein, 118 aa, artificial sequence
  TCR alpha chain variable region
QKEVEQDPGPLSVPEGAIVSLNCTYSNSAFLWPAQFMWYRQYSRKGPELLMYQDMRISSGNKEDGRFTAQVDKSSKYISLFIRDSQPSDSATYLCAMRGDSSYKLIFGSGTRLLVRPD

SEQ ID NO: 55 - protein, 118 aa, artificial sequence
  TCR alpha chain variable region
QKEVEQDPGPLSVPEGAIVSLNCTYSNSAFLWPAQFMWYRQYSRKGPELLMYTYMGSFYGNKEDGRFTAQVDKSSKYISLFIRDSQPSDSATYLCAMRGDSSYKLIFGSGTRLLVRPD

SEQ ID NO: 56 - protein, 118 aa, artificial sequence
  TCR alpha chain variable region
QKEVEQDPGPLSVPEGAIVSLNCTYSNSAFLWPAQFMWYRQYSRKGPELLMYLPQPWSSGNKEDGRFTAQVDKSSKYISLFIRDSQPSDSATYLCAMRGDSSYKLIFGSGTRLLVRPD

SEQ ID NO: 57 - protein, 118 aa, artificial sequence
  TCR alpha chain variable region
QKEVEQDPGPLSVPEGAIVSLNCTYSERQYDAFQYFMWYRQYSRKGPELLMYQDMRISSGNKEDGRFTAQVDKSSKYISLFIRDSQPSDSATYLCAMRGDSSYKLIFGSGTRLLVRPD

SEQ ID NO: 58 - protein, 118 aa, artificial sequence
  TCR alpha chain variable region
QKEVEQDPGPLSVPEGAIVSLNCTYSERQYDAFQYFMWYRQYSRKGPELLMYTYMGSFYGNKEDGRFTAQVDKSSKYISLFIRDSQPSDSATYLCAMRGDSSYKLIFGSGTRLLVRPD

SEQ ID NO: 59 - protein, 118 aa, artificial sequence
  TCR alpha chain variable region
QKEVEQDPGPLSVPEGAIVSLNCTYSERQYDAFQYFMWYRQYSRKGPELLMYLPQPWSSGNKEDGRFTAQVDKSSKYISLFIRDSQPSDSATYLCAMRGDSSYKLIFGSGTRLLVRPD

SEQ ID NO: 60 - protein, 116 aa, artificial sequence
  TCR beta chain variable region
DAGVIQSPRHEVTEMGQEVTLRCKPISGHDYLFWYRQTMMRGLELLIYFRWGEVIDDSGMPEDRFSAKMPNASFSTLKIQPSEPRDSAVYFCASSLWEDLQRGIQYFGAGTRLSVL

SEQ ID NO: 61 - protein, 116 aa, artificial sequence
  TCR beta chain variable region
DAGVIQSPRHEVTEMGQEVTLRCKPISGHDYLFWYRQTMMRGLELLIYFRWGEIIDDSGMPEDRFSAKMPNASFSTLKIQPSEPRDSAVYFCASSLWEDLQRGIQYFGAGTRLSVL

SEQ ID NO: 62 - protein, 116 aa, artificial sequence
  TCR beta chain variable region
DAGVIQSPRHEVTEMGQEVTLRCKPISGHDYLFWYRQTMMRGLELLIYFRWGETIDDSGMPEDRFSAKMPNASFSTLKIQPSEPRDSAVYFCASSLWEDLQRGIQYFGAGTRLSVL

SEQ ID NO: 63 - protein, 116 aa, artificial sequence
  TCR beta chain variable region
DAGVIQSPRHEVTEMGQEVTLRCKPISGHDYLFWYRQTMMRGLELLIYFHWGQSIDDSGMPEDRFSAKMPNASFSTLKIQPSEPRDSAVYFCASSLWEDLQRGIQYFGAGTRLSVL

SEQ ID NO: 64 - protein, 116 aa, artificial sequence
  TCR beta chain variable region
DAGVIQSPRHEVTEMGQEVTLRCKPISGHDYLFWYRQTMMRGLELLIYFRWGVPIDDSGMPEDRFSAKMPNASFSTLKIQPSEPRDSAVYFCASSLWEDLQRGIQYFGAGTRLSVL

SEQ ID NO: 65 - protein, 116 aa, artificial sequence
  TCR beta chain variable region
DAGVIQSPRHEVTEMGQEVTLRCKPISGHDYLFWYRQTMMRGLELLIYFRWSEPIDDSGMPEDRFSAKMPNASFSTLKIQPSEPRDSAVYFCASSLWEDLQRGIQYFGAGTRLSVL

SEQ ID NO: 66 - protein, 116 aa, artificial sequence
  TCR beta chain variable region
DAGVIQSPRHEVTEMGQEVTLRCKPISGHDYLFWYRQTMMRGLELLIYFRFGEVIDDSGMPEDRFSAKMPNASFSTLKIQPSEPRDSAVYFCASSLWEDLQRGIQYFGAGTRLSVL

SEQ ID NO: 67 - protein, 116 aa, artificial sequence
  TCR beta chain variable region
DAGVIQSPRHEVTEMGQEVTLRCKPISGHDYLFWYRQTMMRGLELLIYFKWSEVIDDSGMPEDRFSAKMPNASFSTLKIQPSEPRDSAVYFCASSLWEDLQRGIQYFGAGTRLSVL

SEQ ID NO: 68 - protein, 116 aa, artificial sequence
  TCR beta chain variable region
DAGVIQSPRHEVTEMGQEVTLRCKPISGHDYLFWYRQTMMRGLELLIYFRFGVAIDDSGMPEDRFSAKMPNASFSTLKIQPSEPRDSAVYFCASSLWEDLQRGIQYFGAGTRLSVL

SEQ ID NO: 69 - protein, 116 aa, artificial sequence
  TCR beta chain variable region
DAGVIQSPRHEVTEMGQEVTLRCKPISGHDYLFWYRQTMMRGLELLIYFRWAEVIDDSGMPEDRFSAKMPNASFSTLKIQPSEPRDSAVYFCASSLWEDLQRGIQYFGAGTRLSVL

SEQ ID NO: 70 - protein, 116 aa, artificial sequence
  TCR beta chain variable region
DAGVIQSPRHEVTEMGQEVTLRCKPISGHDYLFWYRQTMMRGLELLIYFRWGTAIDDSGMPEDRFSAKMPNASFSTLKIQPSEPRDSAVYFCASSLWEDLQRGIQYFGAGTRLSVL

SEQ ID NO: 71 - protein, 116 aa, artificial sequence
  TCR beta chain variable region
DAGVIQSPRHEVTEMGQEVTLRCKPISGHDYLFWYRQTMMRGLELLIYFRYGEPIDDSGMPEDRFSAKMPNASFSTLKIQPSEPRDSAVYFCASSLWEDLQRGIQYFGAGTRLSVL

SEQ ID NO: 72 - protein, 116 aa, artificial sequence
  TCR beta chain variable region
DAGVIQSPRHEVTEMGQEVTLRCKPISGHDYLFWYRQTMMRGLELLIYFRYGEIIDDSGMPEDRFSAKMPNASFSTLKIQPSEPRDSAVYFCASSLWEDLQRGIQYFGAGTRLSVL

SEQ ID NO: 73 - protein, 116 aa, artificial sequence
  TCR beta chain variable region
DAGVIQSPRHEVTEMGQEVTLRCKPISGHDYLFWYRQTMMRGLELLIYFRYGEAIDDSGMPEDRFSAKMPNASFSTLKIQPSEPRDSAVYFCASSLWEDLQRGIQYFGAGTRLSVL

SEQ ID NO: 74 - protein, 116 aa, artificial sequence
  TCR beta chain variable region
DAGVIQSPRHEVTEMGQEVTLRCKPISGHDYLFWYRQTMMRGLELLIYFMYGYPIDDSGMPEDRFSAKMPNASFSTLKIQPSEPRDSAVYFCASSLWEDLQRGIQYFGAGTRLSVL

SEQ ID NO: 75 - protein, 116 aa, artificial sequence
  TCR beta chain variable region
DAGVIQSPRHEVTEMGQEVTLRCKPISGHDYLFWYRQTMMRGLELLIYFHWGEPIDDSGMPEDRFSAKMPNASFSTLKIQPSEPRDSAVYFCASSLWEDLQRGIQYFGAGTRLSVL

SEQ ID NO: 76 - protein, 116 aa, artificial sequence
  TCR beta chain variable region
DAGVIQSPRHEVTEMGQEVTLRCKPISGHDYLFWYRQTMMRGLELLIYFWRGMAIDDSGMPEDRFSAKMPNASFSTLKIQPSEPRDSAVYFCASSLWEDLQRGIQYFGAGTRLSVL

SEQ ID NO: 77 - protein, 116 aa, artificial sequence
  TCR beta chain variable region
DAGVIQSPRHEVTEMGQEVTLRCKPISGHDYLFWYRQTMMRGLELLIYFHAGYPIDDSGMPEDRFSAKMPNASFSTLKIQPSEPRDSAVYFCASSLWEDLQRGIQYFGAGTRLSVL

SEQ ID NO: 78 - protein, 116 aa, artificial sequence
  TCR beta chain variable region
DAGVIQSPRHEVTEMGQEVTLRCKPISGHDYLFWYRQTMMRGLELLIYFYYGAVIDDSGMPEDRFSAKMPNASFSTLKIQPSEPRDSAVYFCASSLWEDLQRGIQYFGAGTRLSVL

SEQ ID NO: 79 - protein, 116 aa, artificial sequence
  TCR beta chain variable region
DAGVIQSPRHEVTEMGQEVTLRCKPISGHDYLFWYRQTMMRGLELLIYFHWGEAIDDSGMPEDRFSAKMPNASFSTLKIQPSEPRDSAVYFCASSLWEDLQRGIQYFGAGTRLSVL

SEQ ID NO: 80 - protein, 116 aa, artificial sequence
  TCR beta chain variable region
DAGVIQSPRHEVTEMGQEVTLRCKPISGHDYLFWYRQTMMRGLELLIYFRWQEVIDDSGMPEDRFSAKMPNASFSTLKIQPSEPRDSAVYFCASSLWEDLQRGIQYFGAGTRLSVL

SEQ ID NO: 81 - protein, 116 aa, artificial sequence
  TCR beta chain variable region
DAGVIQSPRHEVTEMGQEVTLRCKPISGHDYLFWYRQTMMRGLELLIYFAYGVAIDDSGMPEDRFSAKMPNASFSTLKIQPSEPRDSAVYFCASSLWEDLQRGIQYFGAGTRLSVL

SEQ ID NO: 82 - protein, 116 aa, artificial sequence
  TCR beta chain variable region
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSTWADLQRGIQYFGAGTRLSVL

SEQ ID NO: 83 - protein, 116 aa, artificial sequence
  TCR beta chain variable region
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSSWADLQRGIQYFGAGTRLSVL

SEQ ID NO: 84 - protein, 116 aa, artificial sequence
  TCR beta chain variable region
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSSWGDLQRGIQYFGAGTRLSVL

SEQ ID NO: 85 - protein, 116 aa, artificial sequence
  TCR beta chain variable region
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSTWRDLQRGIQYFGAGTRLSVL

SEQ ID NO: 86 - protein, 116 aa, artificial sequence
  TCR beta chain variable region
DAGVIQSPRHEVTEMGQEVTLRCKPISGHDYLFWYRQTMMRGLELLIYFRWSEPIDDSGMPEDRFSAKMPNASFSTLKIQPSEPRDSAVYFCASSTWADLQRGIQYFGAGTRLSVL

SEQ ID NO: 87 - protein, 116 aa, artificial sequence
  TCR beta chain variable region
DAGVIQSPRHEVTEMGQEVTLRCKPISGHDYLFWYRQTMMRGLELLIYFRWSEPIDDSGMPEDRFSAKMPNASFSTLKIQPSEPRDSAVYFCASSTWRDLQRGIQYFGAGTRLSVL

SEQ ID NO: 88 - protein, 116 aa, artificial sequence
  TCR beta chain variable region
DAGVIQSPRHEVTEMGQEVTLRCKPISGHDYLFWYRQTMMRGLELLIYFKWSEVIDDSGMPEDRFSAKMPNASFSTLKIQPSEPRDSAVYFCASSTWADLQRGIQYFGAGTRLSVL

SEQ ID NO: 89 - protein, 116 aa, artificial sequence
  TCR beta chain variable region
DAGVIQSPRHEVTEMGQEVTLRCKPISGHDYLFWYRQTMMRGLELLIYFKWSEVIDDSGMPEDRFSAKMPNASFSTLKIQPSEPRDSAVYFCASSTWRDLQRGIQYFGAGTRLSVL

SEQ ID NO: 90 - protein, 116 aa, artificial sequence
  TCR beta chain variable region
DAGVIQSPRHEVTEMGQEVTLRCKPISGHDYLFWYRQTMMRGLELLIYFMYGYPIDDSGMPEDRFSAKMPNASFSTLKIQPSEPRDSAVYFCASSTWADLQRGIQYFGAGTRLSVL

SEQ ID NO: 91 - protein, 116 aa, artificial sequence
  TCR beta chain variable region
DAGVIQSPRHEVTEMGQEVTLRCKPISGHDYLFWYRQTMMRGLELLIYFMYGYPIDDSGMPEDRFSAKMPNASFSTLKIQPSEPRDSAVYFCASSTWRDLQRGIQYFGAGTRLSVL

SEQ ID NO: 92 - DNA, 45 bp, Homo sapiens
gaattccatatgcaaaaagaagttgaacaagatcctggaccactc

SEQ ID NO: 93 - DNA, 36 bp, Homo sapiens
ttgtcagtcgacttagagtctctcagctggtacacg

SEQ ID NO: 94 - DNA, 45 bp, Homo sapiens
gaattccatatggatgctggagttattcaatcaccccggcacgag

SEQ ID NO: 95 - DNA, 32 bp, Homo sapiens
tagaaaccggtggccaggcacaccagtgtggc

(19) United States
(12) Patent Application Publication
(10) Pub. No.: US 2016/0318988 A1
(43) Pub. Date: Nov. 3, 2016

(54) T CELL RECEPTOR BINDING TO ALWGPDPAAA, DERIVED FROM HUMAN PRE-PRO INSULIN (PPI) PROTEIN

(71) Applicants: IMMUNOCORE LIMITED, Abingdon Oxfordshire (GB); ADAPTIMMUNE LIMITED, Abingdon Oxfordshire (GB)

(72) Inventor: Andrew Alexander KNOX, Abingdon Oxfordshire (GB)

(21) Appl. No.: 15/181,466

(22) Filed: Jun. 14, 2016

Related U.S. Application Data
(63) Continuation-in-part of application No. PCT/GB2014/053625, filed on Dec. 5, 2014.
(60) Provisional application No. 61/917,607, filed on Dec. 18, 2013.

(30) Foreign Application Priority Data
Dec. 18, 2013 (GB) 1322430.8

(51) Int. Cl. C07K 14/725; C07K 14/62; C07K 14/74

(52) U.S. Cl. CPC C07K 14/7051; C07K 14/70539; C07K 14/62

(57) ABSTRACT

The present invention provides a T cell receptor (TCR) having the property of binding to ALWGPDPAAA (derived from human pre-pro insulin (PPI) protein) HLA-A*02 complex and comprising a TCR alpha chain variable domain and a TCR beta chain variable domain. Also provided are nucleic acids encoding the TCR and cells engineered to present the TCR. Therapeutic agents based on TCRs of the invention can be used for the purpose of delivering immunosuppressive agents to beta cells in order to prevent their destruction by CD8⁺ T cells.

### RELATED APPLICATIONS AND INCORPORATION BY REFERENCE

This application is a Continuation-In-Part application of International Patent Application Serial No. PCT/GB2014/053625 filed Dec. 5, 2014, which published as PCT Publication No. WO 2015/092362 on Jun. 25, 2015, which claims benefit of United Kingdom Patent Application Serial No. GB 1322430.8 filed Dec. 18, 2013 and U.S. Provisional Application No. 61/917,607 filed Dec. 18, 2013.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE LISTING

The instant application contains a Sequence Listing which has been submitted electronically in ASCII format and is hereby incorporated by reference in its entirety. Said ASCII copy, created on Jun. 9, 2016, is named 49047_01_2019 SL.txt and is 104,472 bytes in size.

### FIELD OF THE INVENTION

The present invention relates to T cell receptors (TCRs) which bind the ALWGPDPAAA peptide (derived from human pre-pro insulin (PPI) protein) presented as a peptide-HLA-A*02 complex. The TCRs have improved binding affinities for, and/or binding half-lives for, the peptide HLA complex, compared to the reference PPI TCR described below. The invention also provides T cells transfected with PPI TCRs of the invention, as well as soluble PPI TCRs fused to immunosuppressive agents. Such reagents are useful for the treatment of autoimmune diseases such as diabetes.

### BACKGROUND OF THE INVENTION

Type 1 diabetes mellitus (T1DM) is an auto-immune disease characterised by metabolic dysfunction, most notably dysregulation of glucose metabolism, accompanied by characteristic long-term vascular and neurological complications. T1DM is one of the most common autoimmune diseases, affecting one in 250 individuals in the US where there are approximately 10,000 to 15,000 new cases reported each year, and the incidence is rising. The highest prevalence of T1DM is found in northern Europe, where more than 1 in every 150 Finns develops T1DM by the age of 15. In contrast, T1DM is less common in black and Asian populations where the frequency is less than half that among the white population.

T1DM is characterised by absolute insulin deficiency, making patients dependent on exogenous insulin for survival. Prior to the acute clinical onset of T1DM with symptoms of hyperglycaemia there is a long asymptomatic preclinical period, during which insulin-producing beta cells are progressively destroyed. The autoimmune destruction of beta cells (β cells) is associated with lymphocytic infiltration. In addition, abnormalities in the presentation of MHC Class I antigens on the cell surface have been identified in both animal models and in human T1DM. This immune abnormality may explain why humans become intolerant of self-antigens although it is not clear why only beta cells are preferentially destroyed.

There is ample evidence that CD8⁺ T cells are involved in the disease process that leads to T1DM (Liblau Immunity. 2002 July; 17(1):1-6). Histological analysis of the islets in an affected individual shows infiltration by CD8⁺ T cells (Bottazzo, et al. 1985 N. Engl. J. Med. 313:353-360). In animal models of T1DM, the disease process may be transferred from a diseased animal to a healthy animal using CD8⁺ T cells. There is a genetic association between the development of T1DM and certain HLA class I molecules that are critical for CD8⁺ target recognition (Todd, et al. 2007 Nat. Genet. 39:857-864 and Marron, et al. 2002 Proc. Natl. Acad. Sci. U.S.A. 99:13753-13758.). Finally, activated CD8⁺ T cells are present in the circulation of high-risk subjects who develop T1DM (Skowera, et al. 2008 J Clin Invest. 118:3390-402).

Antigen-specific immunotherapy of type 1 diabetes in the early, post-onset period has the potential to halt disease progression and preserve remaining islet cell function. A safe immunotherapy could also be considered for the protection of islet allografts and for prophylaxis where strong genetic predisposition to type I diabetes is present. Islet beta cells are naturally protected from pathogenic T cells by Foxp3 expressing regulatory CD4⁺ T cells (Treg) (see Wildin et al., (2001) Nat Genet. 27 (1): 18-20) and it is established that protection mediated by adoptively transferred T cells requires recognition of an islet cell antigen (see Tonkin et al., (2008) J Immunol. 181 (7): 4516-22).

A number of diabetes-specific human auto-reactive CD8⁺ T cells have been isolated from diseased individuals (Skowera, et al. 2008 J Clin Invest. 118:3390-402 and Lieberman et al. Proc Natl Acad Sci U.S.A. 2003 Jul. 8; 100(14):8384-8). These T cells bear T cell receptors (TCRs) which primarily recognise peptide epitopes of β-cell antigens such as pre-pro-insulin (PPI). The ALWGPDPAAA₁₅₋₂₄ (SEQ ID No: 1) peptide is one such peptide derived from the signal sequence of human PPI (Skowera, et al. 2008 J Clin Invest. 118:3390-402 and WO2009004315). The peptide is loaded on to HLA-A*02 molecules and presented on the surface of insulin-producing β cells. Therefore, the ALWGPDPAAA-HLA-A*02 complex provides a human beta cell-specific marker that can be recognised by TCRs.

There is a need to provide new compositions for the diagnosis and treatment of T1DM.

According to a first aspect of the invention, there is provided a T cell receptor (TCR) having the property of binding to ALWGPDPAAA (SEQ ID NO: 1) HLA-A*02 complex and comprising a TCR alpha chain variable domain and a TCR beta chain variable domain,
the alpha chain variable domain comprising an amino acid sequence that has at least 90% identity to the sequence of amino acid residues 1-112 of SEQ ID No: 2, and
the beta chain variable domain comprising an amino acid sequence that has at least 90% identity to the sequence of amino acid residues 1-116 of SEQ ID No: 3,
wherein the alpha chain variable domain has at least one of the following substitutions, with reference to the numbering of SEQ ID NO: 2:—

| Residue number | Substitutions | | | | | | | | |
|---|---|---|---|---|---|---|---|---|---|
| N27 | E | | | | | | | | |
| S28 | Q | P | D | Y | T | R | | | |
| A29 | Y | F | Q | L | G | | | | |
| F30 | A | M | Y | L | I | | | | |
| Q31 | T | S | K | R | Q | W | N | L | G |
| Y32 | R | A | V | P | K | W | Q | | |
| T50 | C | G | I | Q | V | M | L | | |
| Y51 | Q | S | G | P | D | | | | |
| S52 | R | L | S | T | A | G | V | M | |
| S53 | V | R | L | N | Q | P | G | | |
| G54 | C | H | R | Q | S | L | | | |

and/or at least one amino acid insertion,
and/or the beta chain variable domain has at least one of the following substitutions, with reference to the numbering of SEQ ID No: 3:—

| Residue number | Substitutions | | | | | | |
|---|---|---|---|---|---|---|---|
| N50 | R | H | K | M | W | Y | A |
| N51 | W | F | Y | R | A | | |
| N52 | G | S | A | Q | | | |
| V53 | E | Q | T | Y | M | A | |
| P54 | V | I | T | S | A | | |
| L96 | T | S | | | | | |
| E98 | A | G | R | | | | |
| K99 | D | | | | | | |
| A101 | Q | | | | | | |
| K102 | R | | | | | | |
| N103 | G | | | | | | |

Therapeutic agents based on TCR molecules of the invention can be used for the purpose of delivering immunosuppressive agents to beta cells in order to prevent their destruction by CD8⁺ T cells. Such immunosuppressive agents include antibody fragments or cytokines.

TCRs which target the ALWGPDPAAA-HLA-A*02 complex can also be used in the treatment process known as adoptive therapy. It is known that T regulatory cells (Tregs) transfected with MHC class I restricted TCRs can produce enhanced suppression of T effectors cells compared with non-transfected Tregs (Plesa et al. 2012 Blood. 119(15):3420-3430) and that such cells have significant potential in the treatment of autoimmune diseases (Wright et al. 2011 Expert Rev Clin Immunol. 7(2):213-25).

Regulatory T cells (Treg) constitute a small proportion (5 to 10%) of the total population of CD4⁺ T lymphocytes (Powrie et al., (2003) Science 299 (5609): 1030-1). Regulatory T cells are characterized by the constitutive expression of CD25 and the Foxp3 transcription factor. Experiments in rodents where Treg cells have been reduced or functionally altered have shown the spontaneous development of various autoimmune diseases including autoimmune thyroiditis, gastritis and type 1 diabetes (Hori et al., (2003) Science 299 (5609): 1057-61).

Levels of CD4⁺CD25⁺ Treg cells have been shown to be lower in NOD mice, a non-obese diabetes mouse spontaneously developing T1DM, and in patients with T1DM compared to normal controls (Wu et al., (2002) Proc Natl Acad Sci USA 99(19): 12287-92). The injection of CD4⁺CD25⁺ Treg cells into NOD mice can be used to prevent T1DM (Wu et al., (2002) Proc Natl Acad Sci USA 99(19): 12287-92). The NOD mouse model serves as a prototypic model for human autoimmunity as NOD mice develop spontaneous diabetes, which closely mirrors many features of T1D in humans, such as hyperglycemia and presence of autoantibodies directed against islet cells (Sgouroudis et al., (2009) Diabetes Metab Res Rev 25(3): 208-18).

The low frequency of natural Tregs is an important limitation to their therapeutic use. The forkhead/winged helix transcription factor Foxp3 is believed to be a master promoter of regulatory T cell differentiation (Hori et al., (2003) Science 299 (5609): 1057-61). Ectopic expression of Foxp3 converts naïve CD4⁺CD25⁻ T cells into cells with the phenotypic and functional characteristics of regulatory T cells (Hori et al., (2003) Science 299 (5609): 1057-61) making larger numbers of Tregs available for therapeutic use.

It has become clear that antigen-specificity of Tregs is required for a successful suppression of inflammation by Treg adoptive transfer (Tonkin et al., (2008) J Immunol. 181 (7): 4516-22). Additionally, Jaeckel et al., (2005 DIABETES 54: 306-310) found that retroviral transduction of polyclonal CD4⁺ T cells with Foxp3 was not effective in interfering with established type 1 diabetes in vivo. However, administration of Foxp3-transduced T cells with specificity for an islet antigen stabilised and reversed disease in mice with recent-onset diabetes.

Tregs, as CD4⁺ cells, recognise antigens presented by MHC class II which is only expressed on antigen presenting cells (APCs). The destruction of islets cells occurs in diabetes patients probably because of the small repertoire of Tregs available which are restricted to MHC class II-epitopes. MHC class I is expressed on virtually all somatic cells and islet beta cells are likely to have the highest density of diabetes-specific antigen-class I MHC complex. Engineering a new type of Tregs by combining the specificity for such antigen-class I MHC complexes and the suppressor phenotype of Treg could enable such modified Tregs to exercise optimal control over the pro-inflammatory environment which otherwise supports the destruction of the islet cells.

TCRs of the invention may also be used as diagnostic reagents to detect cells presenting the ALWGPDPAAA-HLA-A*02 complex. In this case the TCRs may be fused to a detectable label.

To ensure effective targeting of ALWGPDPAAA-HLA-A*02 presenting β cells, TCRs of the present invention have an improved binding affinity for, and/or binding half-life for, the peptide HLA complex, compared to the reference PPI TCR described below. It is desirable that certain TCRs of the invention, such as those used to deliver therapeutic agents or in diagnosis, have a high affinity and/or a slow off-rate for the peptide-HLA complex.

TCRs are described using the International Immunogenetics (IMGT) TCR nomenclature, and links to the IMGT public database of TCR sequences. Native alpha-beta heterodimeric TCRs have an alpha chain and a beta chain. Broadly, each chain comprises variable, joining and constant regions, and the beta chain also usually contains a short diversity region between the variable and joining regions, but this diversity region is often considered as part of the joining region. Each variable region comprises three CDRs (Complementarity Determining Regions) embedded in a framework sequence, one being the hypervariable region named CDR3. There are several types of alpha chain variable (Vα) regions and several types of beta chain variable (Vβ) regions distinguished by their framework, CDR1 and CDR2 sequences, and by a partly defined CDR3 sequence. The Vα types are referred to in IMGT nomenclature by a unique TRAV number. Thus “TRAV12-3” defines a TCR Vα region having unique framework and CDR1 and CDR2 sequences, and a CDR3 sequence which is partly defined by an amino acid sequence which is preserved from TCR to TCR but which also includes an amino acid sequence which varies from TCR to TCR. In the same way, “TRBV12-4” defines a TCR VP region having unique framework and CDR1 and CDR2 sequences, but with only a partly defined CDR3 sequence.

The joining regions of the TCR are similarly defined by the unique IMGT TRAJ and TRBJ nomenclature, and the constant regions by the IMGT TRAC and TRBC nomenclature. The beta chain diversity region is referred to in IMGT nomenclature by the abbreviation TRBD, and, as mentioned, the concatenated TRBD/TRBJ regions are often considered together as the joining region.

The α and β chains of αβ TCR's are generally regarded as each having two “domains”, namely variable and constant domains. The variable domain consists of a concatenation of variable region and joining region. In the present specification and claims, the term “TCR alpha variable domain” therefore refers to the concatenation of TRAV and TRAJ regions, and the term TCR alpha constant domain refers to the extracellular TRAC region, or to a C-terminal truncated TRAC sequence. Likewise the term “TCR beta variable domain” refers to the concatenation of TRBV and TRBD/TRBJ regions, and the term TCR beta constant domain refers to the extracellular TRBC region, or to a C-terminal truncated TRBC sequence.

The unique sequences defined by the IMGT nomenclature are widely known and accessible to those working in the TCR field. For example, they can be found in the IMGT public database. The “T cell Receptor Factsbook”, (2001) LeFranc and LeFranc, Academic Press, ISBN 0-12-441352-8 also discloses sequences defined by the IMGT nomenclature, but because of its publication date and consequent time-lag, the information therein sometimes needs to be confirmed by reference to the IMGT database.

A native PPI TCR has the following alpha chain and beta chain V, J and C gene usage:
Alpha chain—TRAV12-3/TRAJ12/TRAC (the extracellular sequence of the native PPI TCR alpha chain is given in FIG. 1 (SEQ ID NO: 2). The CDRs are defined by amino acids 27-32 (CDR1) 50-55 (CDR2) and 90-100 (CDR3).
Beta chain—TRBV12-4/TRBJ2-4/TRBD2*02/TRBC2 (the extracellular sequence of the native PPI TCR beta chain is given in FIG. 2 (SEQ ID NO: 3). Note the TRBD2 sequence has 2 allelic variants designated in IMGT nomenclature as TRBD2*01 and *02 and the native PPI TCR clone referred to above has the *02 variation. Note also that the absence of a “*” qualifier means that only one allele is known for the relevant sequence. The CDRs are defined by amino acids 27-31 (CDR1), 49-54 (CDR2) and 93-106 (CDR3).

The terms “wild type TCR”, “native TCR”, “wild type PPI TCR”, and “native PPI TCR” are used synonymously herein to refer to this naturally occurring TCR having the extracellular alpha and beta chain SEQ ID NOs: 2 and 3 respectively. An isolated and/or recombinant and/or non-naturally occurring and/or engineered TCR comprising the alpha and beta chain variable domains of SEQ ID NOs: 2 and 3 respectively forms another aspect of the invention. A known PPI TCR is described in Bulek, et al. 2012 Nat Immunol. 13:283-9 and Skowera, et al. 2008 J Clin Invest. 118:3390-402, although relative to a TCR comprising the alpha and beta chain variable domains of SEQ ID NOs: 2 and 3 respectively, this known TCR has Q at position 18 instead of E in the β chain.

For the purpose of providing a reference TCR against which the binding profile of TCRs of the invention may be compared, it is convenient to use the soluble TCR having the extracellular sequence of the native PPI TCR alpha chain given in FIG. 3 (SEQ ID No: 4) and the extracellular sequence of the native PPI TCR beta chain given in FIG. 4 (SEQ ID No: 5). That TCR is referred to herein as the “the reference TCR” or “the reference PPI TCR”. Note that SEQ ID No 4: is the native alpha chain extracellular sequence ID No 2: except that C159 has been substituted for T159 (i.e. T48 of TRAC). Likewise SEQ ID No 5: is the native beta chain extracellular sequence ID No 3: except that that C173 has been substituted for S173 (i.e. S57 of the TRBC2 constant region), A191 has been substituted for C191 and D205 has been substituted for N205. These cysteine substitutions relative to the native alpha and beta chain extracellular sequences enable the formation of an interchain disulfide bond which stabilises the refolded soluble TCR, i.e. the TCR formed by refolding extracellular alpha and beta chains. Use of the stable disulfide linked soluble TCR as the reference TCR enables more convenient assessment of binding affinity and binding half life.

TCRs of the invention may be non-naturally occurring and/or purified and/or engineered. The inventors have surprising found that insertions as well as substitutions in the alpha chain variable domain result in an improved binding affinity/increased half life. TCRs of the invention may have one or more insertions present in the alpha chain variable domain. Additionally or alternatively, they may have one or more insertions present in the beta chain variable domain. The number of inserted amino acids may be in the range of from 1-8, 2-5 and/or may be 1, 2, 3, 4, or 5. It is currently preferred if 2 or 3 amino acids are inserted. Whilst not wishing to be bound by theory, it is believed that the insertions extend the CDRs and increase contact between the CDRs and the peptide-MHC complex by bringing them closer together. TCRs having insertions therein may be suitable for use as therapeutic and/or diagnostic reagents when coupled to a detectable label or therapeutic agent.

The alpha chain variable domain may have at least one amino acid inserted immediately after the residue corresponding to S28, F30, Y32, Y51, S52, S53, G54 and/or D58. Preferably, the alpha chain variable domain may have at least one amino acid inserted immediately after the residue corresponding to S28, Y32, Y51 and/or S53, with reference to the numbering of SEQ ID NO: 2.

In the alpha chain variable domain, the insertion may be one or more of the following, after the indicated residue (with reference to the numbering of SEQ ID NO: 2):

| Residue | | | | | | | | | | | |
|---|---|---|---|---|---|---|---|---|---|---|---|
| number | Inserted residues | | | | | | | | | | |
| S28 | QYD | | | | | | | | | | |
| F30 | DQP | KNP | NQP | | | | | | | | |
| Y32 | PAQ | QL | VL | TQ | PHM | YTA | PQV | PTM | FTR | PQM | NPM |
| Y51 | QPW | MRI | YHQ | TQL | AIT | | | | | | |
| S52 | FK | FQ | HA | | | | | | | | |
| S53 | SFY | LDT | RKN | | | | | | | | |
| G54 | HH | H | HG | TRY | SLD | | | | | | |
| D58 | D | | | | | | | | | | |

The alpha chain variable domain may have an insertion at S28 alone or in combination with an insertion at Y51 or S53 or an insertion at Y32 alone or in combination with an insertion at Y51 or S53. In the alpha chain variable domain, the insertion may be one or more of the following (with reference to the numbering of SEQ ID NO: 2):

| Residue number | Insertion |
|---|---|
| S28 | QYD |
| Y32 | PAQ |
| Y51 | QPW or MRI |
| S53 | SFY |

The insertion may be QYD immediately after S28, with reference to the numbering of SEQ ID NO: 2, optionally with SFY additionally inserted immediately after S53 with reference to the numbering of SEQ ID NO: 2. Alternatively, the insertion may be PAQ immediately after Y32 and SFY immediately after S53, with reference to the numbering of SEQ ID NO: 2

As is known to those skilled in the art, sequences can be compared to each other, typically using sequence alignment programs and/or algorithms that are well known in the art (for example, BLAST, FASTA and MEGALIGN, etc). The person skilled in the art can readily appreciate that, in such alignments, where a mutation contains a residue insertion or deletion, the sequence alignment will introduce a “gap” (typically represented by a dash, or “A”) in the sequence not containing the inserted or deleted residue.

In certain embodiments, there are 2-11 substitutions in one or both variable domains. There may be 2, 3, 4, 5, 6, 7, 8, 9, 10, or 11, substitutions in one or both variable domains. In some embodiments, the α chain variable domain of the TCR of the invention may comprise an amino acid sequence that has at least 90%, at least 91%, at least 92%, at least 93%, at least 94%, at least 95%, at least 96%, at least 97%, at least 98% or at least 99% identity to the sequence of amino acid residues 1-112 of SEQ ID No: 2, provided that the α chain variable domain has at least one of the insertions and/or substitutions outlined above. In some embodiments, the β chain variable domain of the TCR of the invention may comprise an amino acid sequence that has at least 90%, at least 91%, at least 92%, at least 93%, at least 94%, at least 95%, at least 96%, at least 97%, at least 98% or at least 99% identity to the sequence of amino acid residues 1-116 of SEQ ID No: 3, provided that the β chain variable domain has at least one of the substitutions outlined above.

Further embodiments of the invention are provided by TCRs comprising one of the mutated alpha chain variable region amino acid sequences shown in FIGS. 6 and 7 (SEQ ID Nos: 8-59); and/or the mutated beta chain variable region amino acid sequences shown in FIG. 8 (SEQ ID Nos: 60-91). Specific embodiments of the invention are provided by TCRs comprising one of the mutated alpha chain variable region amino acid sequences shown in SEQ ID Nos: 32, 55, 56, 57, 58 and 59; and/or the mutated beta chain variable region amino acid sequence shown in SEQ ID No: 90.

Insertions and substitutions can be carried out using any appropriate method including, but not limited to, those based on polymerase chain reaction (PCR), restriction enzyme-based cloning, or ligation independent cloning (LIC) procedures. These methods are detailed in many of the of the standard molecular biology texts. For further details regarding polymerase chain reaction (PCR) and restriction enzyme-based cloning, see Sambrook & Russell, (2001) Molecular Cloning—A Laboratory Manual (3rd Ed.) CSHL Press. Further information on ligation independent cloning (LIC) procedures can be found in Rashtchian, (1995) Curr Opin Biotechnol 6(1): 30-6

Also within the scope of the invention are phenotypically silent variants of any TCR disclosed herein. As used herein the term “phenotypically silent variants” is understood to refer to those TCRs which have a K_{D} and/or binding half-life for the ALWGPDPAAA (SEQ ID No: 1) HLA-A*02 complex within the ranges of K_{D}s and binding half-lives described below. For example, as is known to those skilled in the art, it may be possible to produce TCRs that incorporate changes in the constant and/or variable domains thereof compared to those detailed above without altering the affinity for the interaction with the ALWGPDPAAA (SEQ ID No: 1) HLA-A*02 complex. Such trivial variants are included in the scope of this invention. Those TCRs in which one or more conservative substitutions have been made also form part of this invention.

As will be obvious to those skilled in the art, it may be possible to truncate the sequences provided at the C-terminus and/or N-terminus thereof, by 1, 2, 3, 4, 5 or more residues, without substantially affecting the binding characteristics of the TCR. All such trivial variants are encompassed by the present invention.

The TCRs of the invention have the property of binding the ALWGPDPAAA (SEQ ID No: 1) HLA-A*02 complex. Certain TCRs of the invention have been found to specifically bind cells which present this epitope, and are thus particularly suitable as targeting vectors for delivery of therapeutic agents or detectable labels to cells and tissues displaying those epitopes. Specificity in the context of TCRs of the invention relates to their ability to recognise PPI antigen positive HLA-A*02 positive target cells whilst having minimal ability to recognise antigen negative targets cells, particularly non-cancerous human cells.

Certain TCRs of the invention have been found to be highly suitable for use in adoptive therapy. Such TCRs may have a K_{D} for the complex of less than the 200 μM, for example from about 0.1 μM to about 100 μM and/or have a binding half-life (T½) for the complex in the range of from about 3 seconds to about 12 minutes. In some embodiments, TCRs of the invention may have a K_{D} for the complex of from about 0.5 μM to about 50 μM, about 1 μM to about 20 μM or about 2 μM to about 10 μM.

Certain TCRs of the invention have been found to be highly suitable for use as therapeutic and/or diagnostic reagents when coupled to a detectable label or therapeutic agent. Such TCRs may have a K_{D} for the complex in the range of from about 10 pM to about 200 nM and a T½ of about 10 minutes to about 60 hours. In some embodiments, TCRs of the invention may have a K_{D} for the complex of from about 20 pM to about 100 nM, from about 50 pM to about 1 nM, from about 100 pM to about 0.8 nM, from about 200 pM, to about 0.7 nM.

The alpha chain variable domain of TCRs suitable for use as therapeutic and/or diagnostic reagents may have at least one of the following substitutions, with reference to the numbering of SEQ ID NO: 2:

| Residue number | Substitutions |
|---|---|
| N27 | E |
| S28 | R |
| Q31 | L |
| Y32 | W |
| T50 | L or Q |
| Y51 | P or D |
| S52 | M |
| S53 | G |

and/or the beta chain variable domain may have has at least one of the following substitutions, with reference to the numbering of SEQ ID No: 3:—

| Residue number | Substitutions |
|---|---|
| N50 | M |
| N51 | Y |
| N52 | G |
| V53 | Y |
| L96 | T |
| E98 | A |
| K99 | D |
| A101 | Q |
| K102 | R |
| N103 | G |

The alpha chain variable domain of such TCRs may have at least one of the following substitutions, with reference to the numbering of SEQ ID NO: 2:

| Residue number | Substitutions |
|---|---|
| N27 | E |
| S28 | R |
| S52 | M |
| S53 | G |

and/or the beta chain variable domain may have at least one of the following substitutions, with reference to the numbering of SEQ ID No: 3:—

| Residue number | Substitutions |
|---|---|
| N50 | M |
| N51 | Y |
| N52 | G |
| V53 | Y |
| L96 | T |
| E98 | A |
| K99 | D |
| A101 | Q |
| K102 | R |
| N103 | G |

In these alpha chain variable domains, there may be at least one amino acid inserted immediately after the residue corresponding to S28, Y32, Y51 and/or S53, with reference to the numbering of SEQ ID NO:2. These alpha chain variable domains may have an insertion at S28 alone or in combination with an insertion at Y51 or S53 or an insertion at Y32 alone or in combination with an insertion at Y51 or S53. The insertion may be one or more of the following (with reference to the numbering of SEQ ID NO: 2):

| Residue number | Insertion |
|---|---|
| S28 | QYD |
| Y32 | PAQ |
| Y51 | QPW or MRI |
| S53 | SFY |

The insertion may be QYD immediately after S28 and SFY immediately after S53, with reference to the numbering of SEQ ID NO: 2. Alternatively, the insertion may be PAQ immediately after Y32 and SFY immediately after S53, with reference to the numbering of SEQ ID NO: 2. The TCR may comprise one of the mutated alpha chain variable region amino acid sequences shown in SEQ ID Nos: 32, 55, 56, 57, 58 and 59; and/or the mutated beta chain variable region amino acid sequence shown in SEQ ID No: 90.

Binding affinity (inversely proportional to the equilibrium constant K_{D}) and binding half-life (expressed as T½) can be determined by any appropriate method. It will be appreciated that doubling the affinity of a TCR results in halving the K_{D}. T½ is calculated as ln 2 divided by the off-rate (k_{off}). Therefore, doubling of T½ results in a halving in k_{off}. K_{D} and k_{off} values for TCRs are usually measured for soluble forms of the TCR, i.e. those forms which are truncated to remove cytoplasmic and transmembrane domain residues. Therefore it is to be understood that a given TCR meets the requirement that it has a binding affinity for, and/or a binding half-life for, the ALWGPDPAAA HLA-A*02 complex if a soluble form of that TCR meets that requirement. Preferably the binding affinity or binding half-life of a given TCR is measured several times, for example 3 or more times, using the same assay protocol and an average of the results is taken. In a preferred embodiment these measurements are made using the Surface Plasmon Resonance (BIAcore) method of Example 3 herein. The reference ALWGPDPAAA HLA-A*02 TCR has a K_{D} of approximately 287 μM as measured by that method.

The TCRs of the invention may be αβ heterodimers or may be in single chain format. Single chain formats include αβ TCR polypeptides of the type: Vα-L-Vβ, Vβ-L-Vα, Vα-Cα-L-Vβ, Vα-L-Vβ-Cβ or Vα-Cα-L-Vβ-Cβ, wherein Vα and Vβ are TCR α and β variable regions respectively, Cα and Cβ are TCR α and β constant regions respectively, and L is a linker sequence. For use as a targeting agent for delivering therapeutic agents to the antigen presenting cell, the TCR may be in soluble form (i.e. having no transmembrane or cytoplasmic domains). For stability, TCRs of the invention, and preferably soluble αβ heterodimeric TCRs, may have an introduced disulfide bond between residues of the respective constant domains, as described, for example, in WO 03/020763. TCRs of the invention may be isolated, engineered or non-naturally occurring. For use in adoptive therapy, an αβ heterodimeric TCR may, for example, be transfected as full length chains having both cytoplasmic and transmembrane domains.

In some embodiments, the alpha chain variable domain may have at least 96, 97, 98 or 99% sequence identity, or 100% sequence identity, to the amino acid sequence from Q1 to D112 of SEQ ID Nos: 8-59, optionally the subset of 32, 55, 56, 57, 58 and 59, with reference to the numbering of SEQ ID NO: 2. The amino acids underlined in FIGS. 6 and 7 may be invariant.

In some embodiments, the beta chain variable domain may have at least 96, 97, 98 or 99% sequence identity, or 100% sequence identity, to the amino acid sequence from D1 to L116 of SEQ ID Nos: 60-91, optionally 90. The amino acids underlined in FIG. 8 may be invariant.

Alpha-beta heterodimeric TCRs in accordance with the invention may be produced from specific alpha and beta chain combinations as shown in Example 4.

Alpha-beta heterodimeric TCRs of the invention usually comprise an alpha chain TRAC constant domain sequence and a beta chain TRBC1 or TRBC2 constant domain sequence. The alpha and beta chain constant domain sequences may be modified by truncation or substitution to delete the native disulfide bond between Cys4 of exon 2 of TRAC and Cys2 of exon 2 of TRBC1 or TRBC2. The alpha and beta chain constant domain sequences may also be modified by substitution of cysteine residues for Thr 48 of TRAC and Ser 57 of TRBC1 or TRBC2, the said cysteines forming a disulfide bond between the alpha and beta constant domains of the TCR.

As is well-known in the art, TCRs may be subject to post translational modifications. Glycosylation is one such modification, which comprises the covalent attachment of oligosaccharide moieties to defined amino acids in the TCR chain. For example, asparagine residues, or serine/threonine residues are well-known locations for oligosaccharide attachment. The glycosylation status of a particular protein depends on a number of factors, including protein sequence, protein conformation and the availability of certain enzymes. Furthermore, glycosylation status (i.e. oligosaccharide type, covalent linkage and total number of attachments) can influence protein function. Therefore, when producing recombinant proteins, controlling glycosylation is often desirable. Controlled glycosylation has been used to improve antibody based therapeutics. (Jefferis R., Nat Rev Drug Discov. 2009 March; 8(3):226-34.). For soluble TCRs of the invention, glycosylation may be controlled in vivo, by using particular cell lines for example, or in vitro, by chemical modification. Such modifications are desirable, since glycosylation can improve phamacokinetics, reduce immunogenicity and more closely mimic a native human protein (Sinclair A M and Elliott S., Pharm Sci. 2005 August; 94(8):1626-35).

One aspect of the invention provides a multivalent TCR complex comprising at least two TCRs of the invention. In one embodiment, at least two TCR molecules are linked via linker moieties to form multivalent complexes. Preferably the complexes are water soluble, so the linker moiety should be selected accordingly. Furthermore, it is preferable that the linker moiety should be capable of attachment to defined positions on the TCR molecules, so that the structural diversity of the complexes formed is minimised. For example, said TCRs may be linked by a non-peptidic polymer chain or a peptidic linker sequence. A TCR complex of the invention may have a non-peptidic polymer chain or peptidic linker sequence extending between amino acid residues of each TCR which are not located in a variable region sequence of the TCR. Since the complexes of the invention may be for use in medicine, the linker moieties should be chosen with due regard to their pharmaceutical suitability, for example their immunogenicity. Examples of linker moieties which fulfil the above desirable criteria are known in the art, for example the art of linking antibody fragments.

Some soluble TCRs of the invention (or multivalent complexes thereof) are useful for delivering detectable labels or therapeutic agents to the antigen presenting cells and tissues containing the antigen presenting cells. They may therefore be associated (covalently or otherwise) with a detectable label; a therapeutic agent; or a PK modifying moiety (for example by PEGylation).

Detectable labels for diagnostic purposes include for instance, fluorescent labels, radiolabels, enzymes, nucleic acid probes and contrast reagents. Such labelled TCRs or multivalent TCR complexes are useful in a method for detecting a ALWGPDPAAA-HLA-A*02 complex or cells presenting this complex which method comprises contacting a sample to be tested with a TCR or TCR complex of the invention; and detecting binding of the TCR or TCR complex. In tetrameric TCR complexes formed for example, using biotinylated heterodimers, fluorescent streptavidin can be used to provide a detectable label. Such a fluorescently-labelled TCR tetramer is suitable for use in FACS analysis, for example to detect antigen presenting cells carrying the ALWGPDPAAA-HLA-A*02 complex for which these high affinity TCRs are specific.

TCRs of the present invention may be detected by the use of TCR-specific antibodies, in particular monoclonal antibodies.

In a further aspect, a TCR (or multivalent complex thereof) of the present invention may alternatively or additionally be associated with (e.g. covalently or otherwise linked to) a therapeutic agent which may be, for example, an immune effector molecule such as an interleukin or a cytokine. IL-4, IL-10 and IL-13 are example cytokines suitable for association with the TCRs of the present invention.

In a further aspect, the present invention provides a nucleic acid comprising a sequence encoding an α chain variable domain of a TCR of the invention and/or a sequence encoding a β chain variable domain of a TCR of the invention. The nucleic acid may encode a TCR of the invention. In some embodiments, the nucleic acid is cDNA. The nucleic acid may be non-naturally occurring, and/or purified and/or engineered.

In another aspect, the invention provides a vector which comprises nucleic acid of the invention. Preferably the vector is a TCR expression vector.

The vector may be capable of expressing in T cells both Foxp3 and a TCR of the invention. Typically, the TCR α and β chains will be expressed together with a GFP/Foxp3 fusion protein from a tricistronic retroviral vector using viral ribosome skip (2A) and internal ribosome entry sites (IRES). Vectors of this type efficiently convert conventional CD4⁺ T cells into antigen specific regulatory phenotype T cells. Co-delivery of an islet-antigen specific enhanced affinity TCR of the invention and Foxp3 ensures islet specificity is not dissociated from regulatory activity and therefore enables the transfected T cells to exercise optimal control over the pro-inflammatory environment which otherwise supports the destruction of the islet cells.

The invention also provides a cell harbouring a nucleic acid or vector of the invention. The vector may comprise nucleic acid of the invention encoding in a single open reading frame, or two distinct open reading frames, the alpha chain and the beta chain respectively. Another aspect provides a cell harbouring a first expression vector which comprises nucleic acid encoding the alpha chain of a TCR of the invention, and a second expression vector which comprises nucleic acid encoding the beta chain of a TCR of the invention. Such cells are particularly useful in adoptive therapy. The cells of the invention may be isolated and/or recombinant and/or non-naturally occurring and/or engineered.

Since the TCRs of the invention have utility in adoptive therapy, the invention includes a non-naturally occurring, and/or purified and/or or engineered cell, presenting a TCR of the invention. The engineered cell may be a T cell, especially a T regulatory cell (Treg). There are a number of methods suitable for the transfection of T cells with nucleic acid (such as DNA or RNA) encoding the TCRs of the invention (see for example Robbins et al., 2008 J Immunol. 180: 6116-6131 and Plesa et al. 2012 Blood. 119(15):3420-3430). T cells expressing the TCRs of the invention will be suitable for use in adoptive therapy-based treatment of T1DM. As will be known to those skilled in the art, there are a number of suitable methods by which adoptive therapy can be carried out (see for example Rosenberg et al., 2008 Nat Rev Cancer 8(4): 299-308).

In one aspect, the invention provides a pharmaceutical composition which comprises a plurality of regulatory phenotype T cells which recognise a ALWGPDPAAA-HLA-A*02 complex presented on islet cells and one or more pharmaceutical acceptable carriers or excipients, wherein said regulatory phenotype T cells harbour an introduced vector capable of expressing a TCR of the invention, which may be an αβ heterodimeric TCR. Such a composition may be used for the treatment of Type 1 diabetes.

Aspects of the invention which involve TCR transduced T-cells may require αβ heterodimeric TCR-transfected regulatory phenotype T cells which recognise a ALWGPDPAAA-HLA-A*02 complex presented on islet cells. A typical population of CD4⁺ T cells from a given individual will normally comprise about 5-10% of native regulatory T cells. Although the regulatory T cells could be separated from the total population and transfected with the TCR, it is preferred to start with non-regulatory CD4⁺ T cells and introduce a vector capable of expressing Foxp3 to switch them to the regulatory T cell phenotype. Conveniently, the introduced vector capable of expressing Foxp3 is also capable of expressing the said TCR. Usually, but not essentially, the T cells which are transfected with the TCR or with both the TCR and Foxp3, will be taken from the patient to be treated with the compositions of the invention.

For administration to patients, the TCRs, multivalent complexes, nucleic acids, vectors or cells of the invention may be provided in a pharmaceutical composition together with one or more pharmaceutically acceptable carriers or excipients. TCRs, multivalent complexes, nucleic acids, vectors or cells in accordance with the invention will usually be supplied as part of a sterile, pharmaceutical composition which will normally include a pharmaceutically acceptable carrier. This pharmaceutical composition may be in any suitable form, (depending upon the desired method of administering it to a patient). It may be provided in unit dosage form, will generally be provided in a sealed container and may be provided as part of a kit. Such a kit would normally (although not necessarily) include instructions for use. It may include a plurality of said unit dosage forms.

The pharmaceutical composition may be adapted for administration by any appropriate route, preferably a parenteral (including subcutaneous, intramuscular, or preferably intravenous) route. Such compositions may be prepared by any method known in the art of pharmacy, for example by mixing the active ingredient with the carrier(s) or excipient(s) under sterile conditions.

Also provided by the invention are:
a TCR which binds the ALWGPDPAAA peptide presented as a peptide-HLA-A2 complex, a multivalent TCR complex comprising a plurality of such TCRs, a nucleic acid encoding such a TCR or multivalent TCR complex, a vector comprising such a nucleic acid and/or a cell expressing and/or presenting such a TCR, for use in medicine, preferably in a method of treating autoimmune disease;
the use of a TCR which binds the ALWGPDPAAA peptide presented as a peptide-HLA-A2 complex, a multivalent TCR complex comprising a plurality of such TCRs, a nucleic acid encoding such a TCR or multivalent TCR complex, a vector comprising such a nucleic acid and/or a cell expressing and/or presenting such a TCR, in the manufacture of a medicament for the treatment of autoimmune disease;
a method of treating a patient suffering from autoimmune disease, comprising administering to the patient a TCR which binds the ALWGPDPAAA peptide presented as a peptide-HLA-A2 complex, a multivalent TCR complex comprising a plurality of such TCRs, a nucleic acid encoding such a TCR or multivalent TCR complex, a vector comprising such a nucleic acid and/or a cell expressing and/or presenting such a TCR.

It is preferred that the TCR which binds the ALWGPDPAAA peptide presented as a peptide-HLA-A2 complex is a TCR of the invention. Equally, the multivalent TCR complex, nucleic acid, vector and cell may be in accordance with the invention. The autoimmune disease may be type 1 diabetes. The method may comprise adoptive therapy.

Preferred features of each aspect of the invention are as for each of the other aspects mutatis mutandis. The published documents mentioned herein are incorporated to the fullest extent permitted by law. Citation or identification of any document in this application is not an admission that such document is available as prior art to the present invention.

### BRIEF DESCRIPTION OF THE DRAWINGS

The following detailed description, given by way of example, but not intended to limit the invention solely to the specific embodiments described, may best be understood in conjunction with the accompanying drawings.

Reference is made herein to the accompanying drawings in which:

FIG. 1 (SEQ ID NO: 2) gives the amino acid sequence of the extracellular part of the alpha chain of a wild-type PPI-specific TCR with gene usage TRAV12-3/TRAJ12/TRAC.

FIG. 2 (SEQ ID No: 3) gives the amino acid sequence of the extracellular part of the beta chain of a wild-type PPI-specific TCR with gene usage TRBV12-4/TRBJ2-4/TRBD2*02/TRBC2.

FIG. 3 (SEQ ID No: 4) gives the amino acid sequence of the alpha chain of a soluble TCR (referred to herein as the reference TCR). The sequence is the same as that of FIG. 1 except that a cysteine (bold and underlined) is substituted for T159 of SEQ ID No: 1 (i.e. T48 of the TRAC constant region). Complementary determining regions are underlined.

FIG. 4 (SEQ ID No: 5) gives the amino acid sequence of the beta chain of a soluble TCR (referred to herein as the reference TCR). The sequence is the same as that of FIG. 1 except that a cysteine (bold and underlined) is substituted S173 (i.e. S57 of the TRBC2 constant region), and A202 is substituted for C191 and D205 is substituted for N216. Complementary determining regions are underlined.

FIG. 5 (SEQ ID No: 6 and SEQ ID No: 7) gives DNA sequences encoding the TCR alpha and beta chains of FIGS. 3 and 4 respectively (introduced cysteines are shown in bold).

FIGS. 6A-B (SEQ ID Nos: 8-18) gives the amino acid sequences of alpha chain variable domains, containing substitutions, which may be present in the TCRs of the invention.

FIG. 7A-G (SEQ ID No: 19-59) gives the amino acid sequences of further alpha chain variable domains, containing insertions or insertions and substitutions, which may be present in the TCRs of the invention.

FIG. 8A-F (SEQ ID No: 60-91) gives the amino acid sequences of a beta chain variable domains, containing substitutions, which may be present in the TCRs of the invention.

FIG. 9 is a graph showing the results of an experiment in which non-obese diabetic (NOD) mice were injected with TCR-transduced Treg cells.

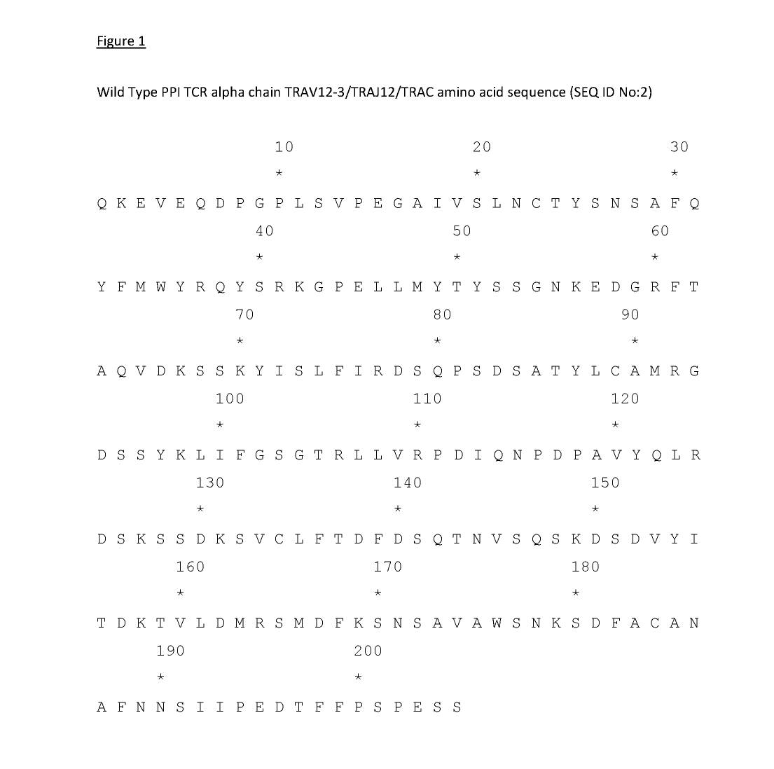

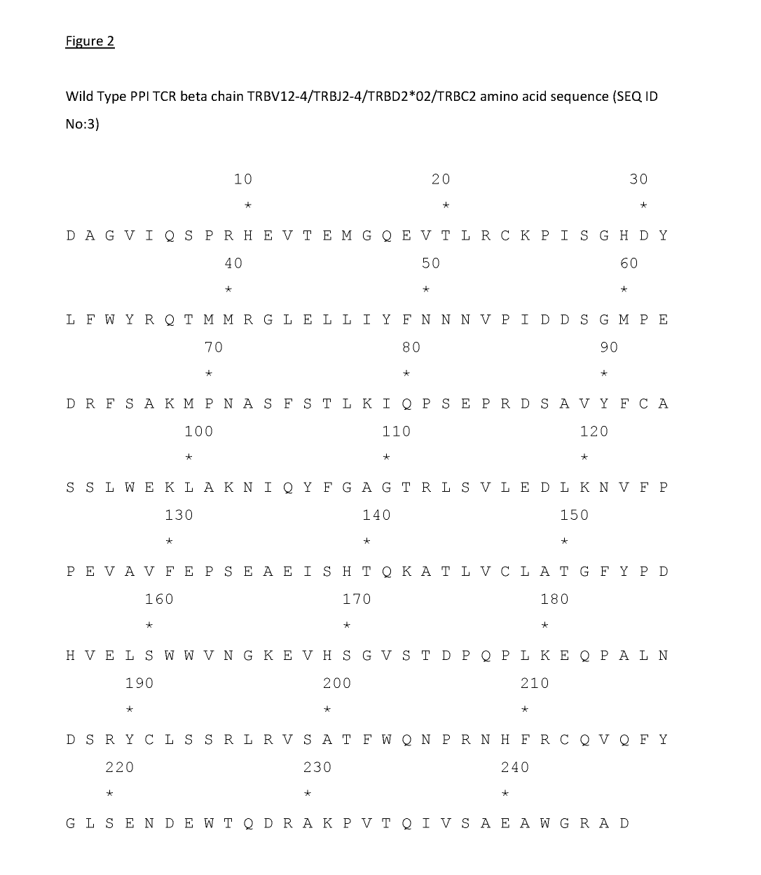

### DETAILED DESCRIPTION OF THE INVENTION

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1

### Cloning of the Reference PPI TCR Alpha and Beta Chain Variable Region Sequences into pEX956 and pEX821-Based Expression Plasmids Respectively

The reference PPI TCR variable alpha and TCR variable beta domains were PCR amplified from total cDNA isolated from a PPI T cell clone (Clone 1E6 from Mark Peakman, King's College London, United Kingdom). In the case of the alpha chain, an alpha chain variable region sequence specific oligonucleotide A1 (primer sequence: gaattccatatgcaaaaagaagttgaacaagatcctggaccactc (SEQ ID No: 92)) which encodes the restriction site NdeI and an alpha chain constant region sequence specific oligonucleotide A2 (primer sequence: ttgtcagtcgacttagagtctctcagctggtacacg (SEQ ID No: 93)) which encodes the restriction site SalI are used to amplify the alpha chain variable domain. In the case of the beta chain, a beta chain variable region sequence specific oligonucleotide B1 (primer sequence: gaattccatatggatgctggagttattcaatcaccccggcacgag (SEQ ID No: 94)) which encodes the restriction site NdeI and a beta chain constant region sequence specific oligonucleotide B2 (primer sequence: tagaaaccggtggccaggcacaccagtgtggc (SEQ ID No: 95)) which encodes the restriction site AgeI are used to amplify the beta chain variable domain.

The alpha and beta variable domains were cloned into pEX956 and pEX821 based expression plasmids respectively containing either Cα or Cβ, by standard methods described in (Molecular Cloning a Laboratory Manual Third edition by Sambrook and Russell). Plasmids were sequenced using an Applied Biosystems 3730xl DNA Analyzer.

The DNA sequences encoding the TCR alpha chain cut with NdeI and SalI were ligated into pEX956+Ca vector, which was cut with NdeI and XhoI. The DNA sequences encoding the TCR beta chain cut with NdeI and AgeI was ligated into separate pEX821+Cb vector, which was also cut with NdeI and AgeI.

Ligated plasmids were transformed into competent E. coli strain XL1-blue cells and plated out on LB/agar plates containing 100 μg/ml ampicillin. Following incubation 10 overnight at 37° C., single colonies are picked and grown in 10 ml LB containing 100 μg/ml ampicillin overnight at 37° C. with shaking. Cloned plasmids were purified using a Miniprep kit (Qiagen) and the plasmids were sequenced using an Applied Biosystems 3730xl DNA Analyzer.

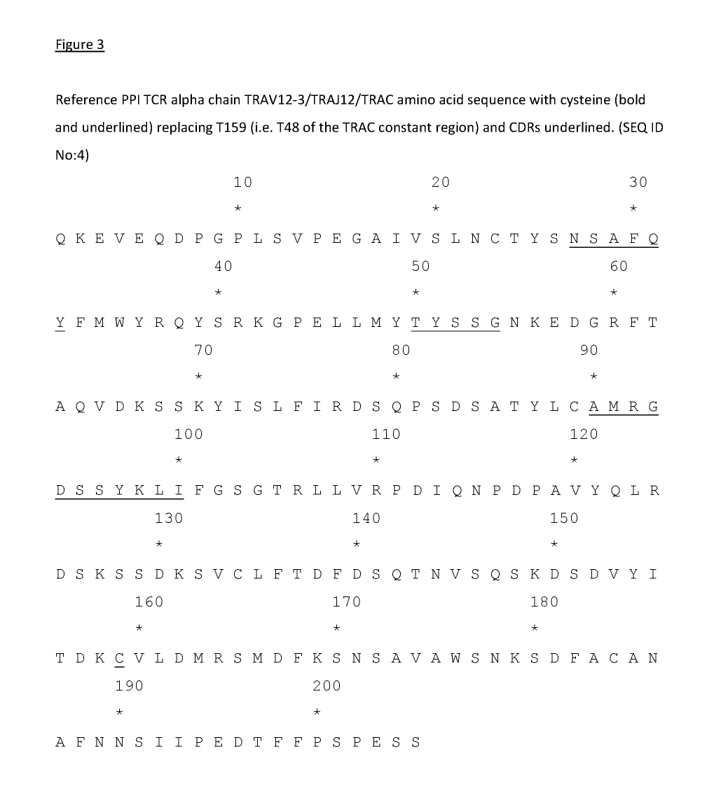

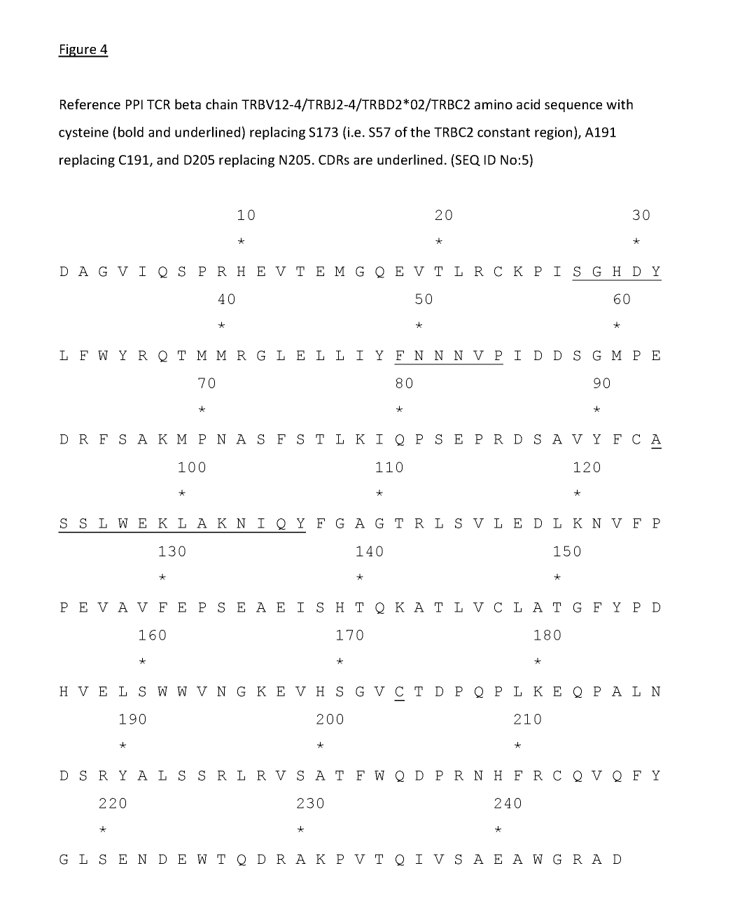

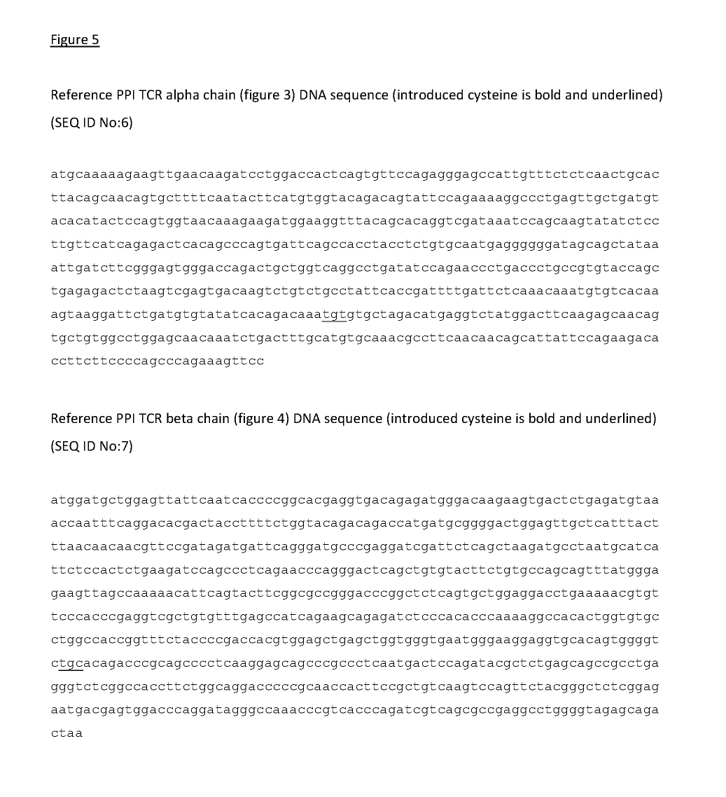

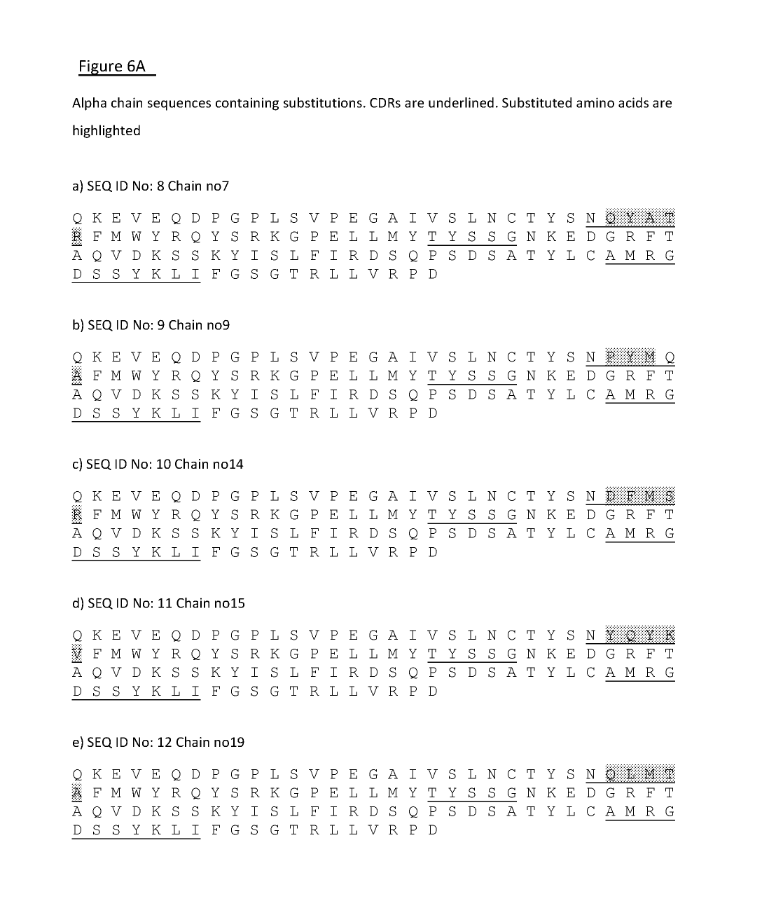

FIGS. 3 and 4 show respectively the reference PPI TCR alpha and beta chain extracellular amino acid sequences (SEQ ID Nos: 4 and 5) produced from the DNA sequences of FIG. 5 (SEQ ID Nos: 6 and 7). Note that, relative to the native TCR, cysteines were substituted in the constant regions of the alpha and beta chains to provide an artificial inter-chain disulphide bond on refolding to form the heterodimeric TCR. The introduced cysteines are shown in bold and underlined.

### Example 2

### Expression, Refolding and Purification of Soluble Reference PPI TCR

The expression plasmids containing the TCR α-chain and β-chain respectively, as prepared in Example 1, were transformed separately into E. coli strain BL21pLysS, and single ampicillin-resistant colonies were grown at 37° C. in TYP (ampicillin 100 μg/ml) medium to OD₆₀₀ of ˜0.6-0.8 before inducing protein expression with 0.5 mM IPTG. Cells were harvested three hours post-induction by centrifugation for 30 minutes at 4000 rpm in a Beckman J-6B. Cell pellets were lysed with 25 ml Bug Buster® (Novagen) in the presence of MgCl₂ and DNaseI. Inclusion body pellets were recovered by centrifugation for 30 minutes at 13000 rpm in a Beckman J2-21 centrifuge. Three detergent washes were then carried out to remove cell debris and membrane components. Each time the inclusion body pellet was homogenised in a Triton buffer (50 mM Tris-HCl pH 8.0, 0.5% Triton-X100, 200 mM NaCl, 10 mM NaEDTA) before being pelleted by centrifugation for 15 minutes at 13000 rpm in a Beckman J2-21. Detergent and salt was then removed by a similar wash in the following buffer: 50 mM Tris-HCl pH 8.0, 1 mM NaEDTA. Finally, the inclusion bodies were divided into 30 mg aliquots and frozen at −70° C. Inclusion body protein yield was quantified by solubilising with 6 M guanidine-HCl and an OD measurement was taken on a Hitachi U-2001 Spectrophotometer. The protein concentration was then calculated using the extinction coefficient.

Approximately 15 mg of TCR β chain and 15 mg of TCR α chain solubilised inclusion bodies were thawed from frozen stocks and diluted into 10 ml of a guanidine solution (6 M Guanidine-hydrochloride, 50 mM Tris HCl pH 8.1, 100 mM NaCl, 10 mM EDTA, 10 mM DTT), to ensure complete chain denaturation. The guanidine solution containing fully reduced and denatured TCR chains was then injected into 0.5 litre of the following refolding buffer: 100 mM Tris pH 8.1, 400 mM L-Arginine, 2 mM EDTA, 5 M Urea. The redox couple (cysteamine hydrochloride and cystamine dihydrochloride) to final concentrations of 6.6 mM and 3.7 mM respectively, were added approximately 5 minutes before addition of the denatured TCR chains. The solution was left for ˜30 minutes. The refolded TCR was dialysed in Spectra/Por® 1 membrane (Spectrum; Product No. 132670) against 10 L H₂O for 18-20 hours. After this time, the dialysis buffer was changed twice to fresh 10 mM Tris pH 8.1 (10 L) and dialysis was continued at 5° C.±3° C. for another ˜8 hours.

Soluble TCR was separated from degradation products and impurities by loading the dialysed refold onto a POROS® 50HQ anion exchange column and eluting bound protein with a gradient of 0-500 mM NaCl in 10 mM Tris pH 8.1 over 50 column volumes using an Akta® purifier (GE Healthcare). Peak fractions were pooled and a cocktail of protease inhibitors (Calbiochem) were added. The pooled fractions were then stored at 4° C. and analysed by Coomassie-stained SDS-PAGE before being pooled and concentrated. Finally, the soluble TCR was purified and characterised using a GE Healthcare Superdex® 75HR gel filtration column pre-equilibrated in PBS buffer (Sigma). The peak eluting at a relative molecular weight of approximately 50 kDa was pooled and concentrated prior to characterisation by BIAcore® surface plasmon resonance analysis.

### Example 3

### Binding Characterisation

### BIAcore Analysis

A surface plasmon resonance biosensor (BIAcore® 3000) can be used to analyse the binding of a soluble TCR to its peptide-MHC ligand. This is facilitated by producing soluble biotinylated peptide-HLA (“pHLA”) complexes which can be immobilised to a streptavidin-coated binding surface (sensor chip). The sensor chips comprise four individual flow cells which enable simultaneous measurement of T-cell receptor binding to four different pHLA complexes. Manual injection of pHLA complex allows the precise level of immobilised class I molecules to be manipulated easily.

Biotinylated class I HLA-A*02 molecules were refolded in vitro from bacterially-expressed inclusion bodies containing the constituent subunit proteins and synthetic peptide, followed by purification and in vitro enzymatic biotinylation (O'Callaghan et al. (1999) Anal. Biochem. 266: 9-15). HLA-A*02-heavy chain was expressed with a C-terminal biotinylation tag which replaces the transmembrane and cytoplasmic domains of the protein in an appropriate construct. Inclusion body expression levels of ˜75 mg/litre bacterial culture were obtained. The MHC light-chain or β2-microglobulin was also expressed as inclusion bodies in E. coli from an appropriate construct, at a level of ˜500 mg/litre bacterial culture.

E. coli cells were lysed and inclusion bodies were purified to approximately 80% purity. Protein from inclusion bodies was denatured in 6 M guanidine-HCl, 50 mM Tris pH 8.1, 100 mM NaCl, 10 mM DTT, 10 mM EDTA, and was refolded at a concentration of 30 mg/litre heavy chain, 30 mg/litre β2m into 0.4 M L-Arginine, 100 mM Tris pH 8.1, 3.7 mM cystamine dihydrochloride, 6.6 mM cysteamine hydrochloride, 4 mg/L of the AFP peptide required to be loaded by the HLA-A*02 molecule, by addition of a single pulse of denatured protein into refold buffer at <5° C. Refolding was allowed to reach completion at 4° C. for at least 1 hour.

Buffer was exchanged by dialysis in 10 volumes of 10 mM Tris pH 8.1. The protein solution was then filtered through a 1.5 μm cellulose acetate filter and loaded onto a POROS® 50HQ anion exchange column (8 ml bed volume). Protein was eluted with a linear 0-500 mM NaCl gradient in 10 mM Tris pH 8.1 using an Akta® purifier (GE Healthcare). HLA-A*02-peptide complex eluted at approximately 250 mM NaCl, and peak fractions were collected, a cocktail of protease inhibitors (Calbiochem) was added and the fractions were chilled on ice.

Biotinylation tagged pHLA molecules were buffer exchanged into 10 mM Tris pH 8.1, 5 mM NaCl using a GE Healthcare fast desalting column equilibrated in the same buffer. Immediately upon elution, the protein-containing fractions were chilled on ice and protease inhibitor cocktail (Calbiochem) was added. Biotinylation reagents were then added: 1 mM biotin, 5 mM ATP (buffered to pH 8), 7.5 mM MgCl₂, and 5 μg/ml BirA enzyme (purified according to O'Callaghan et al. (1999) Anal. Biochem. 266: 9-15). The mixture was then allowed to incubate at room temperature overnight.

The biotinylated pHLA-A*01 molecules were purified using gel filtration chromatography. A GE Healthcare Superdex® 75 HR 10/30 column was pre-equilibrated with filtered PBS and 1 ml of the biotinylation reaction mixture was loaded and the column was developed with PBS at 0.5 ml/min using an Akta® purifier (GE Healthcare). Biotinylated pHLA-A*02 molecules eluted as a single peak at approximately 15 ml. Fractions containing protein were pooled, chilled on ice, and protease inhibitor cocktail was added. Protein concentration was determined using a Coomassie-binding assay (PerBio) and aliquots of biotinylated pHLA-A*02 molecules were stored frozen at −20° C.

The BIAcore® 3000 surface plasmon resonance (SPR) biosensor measures changes in refractive index expressed in response units (RU) near a sensor surface within a small flow cell, a principle that can be used to detect receptor ligand interactions and to analyse their affinity and kinetic parameters. The BIAcore® experiments were performed at a temperature of 25° C., using PBS buffer (Sigma, pH 7.1-7.5) as the running buffer and in preparing dilutions of protein samples. Streptavidin was immobilised to the flow cells by standard amine coupling methods. The pHLA complexes were immobilised via the biotin tag. The assay was then performed by passing soluble TCR over the surfaces of the different flow cells at a constant flow rate, measuring the SPR response in doing so.

### Equilibrium Binding Constant

The above BIAcore® analysis methods were used to determine equilibrium binding constants. Serial dilutions of the disulfide linked soluble heterodimeric form of the reference PPI TCR were prepared and injected at constant flow rate of 5 μl min-1 over two different flow cells; one coated with 1000 RU of specific ALWGPDPAAA HLA-A*02 complex, the second coated with 1000 RU of non-specific HLA-A*02-peptide complex. Response was normalised for each concentration using the measurement from the control cell. Normalised data response was plotted versus concentration of TCR sample and fitted to a non-linear curve fitting model in order to calculate the equilibrium binding constant, K_{D} (Price & Dwek, Principles and Problems in Physical Chemistry for Biochemists (2nd Edition) 1979, Clarendon Press, Oxford). The disulfide linked soluble form of the reference PPI TCR (Example 2) demonstrated a K_{D} of approximately 287 μM. From the same BIAcore data the T½ value was too fast to be measured

### Kinetic Parameters

For high affinity TCRs K_{D} was determined by experimentally measuring the dissociation rate constant, kd, and the association rate constant, ka. The equilibrium constant K_{D} was calculated as kd/ka. TCR was injected over two different cells one coated with 1000 RU of specific ALWGPDPAAA HLA-A*02 complex, the second coated with 1000 RU of non-specific HLA-A1-peptide complex. Flow rate was set at 50 μl/min. Typically 250 μl of TCR at ˜1 μM concentration was injected. Buffer was then flowed over until the response had returned to baseline or >2 hours had elapsed. Kinetic parameters were calculated using BIAevaluation software. The dissociation phase was fitted to a single exponential decay equation enabling calculation of half-life.

### Example 4

### Generation of Improved Affinity PPI TCRs

The reference PPI TCR described in Example 1 was used a template from which to produce the TCRs of the invention having an increased affinity for the ALWGPDPAAA HLA-A*02 complex.

As is known to those skilled in the art, the necessary codon substitutions or codon insertions required to produce these mutated chains can be introduced into the DNA encoding the corresponding wild-type insulin-specific murine TCR chains by site-directed mutagenesis (QuickChange™ Site-Directed Mutagenesis Kit from Stratagene).

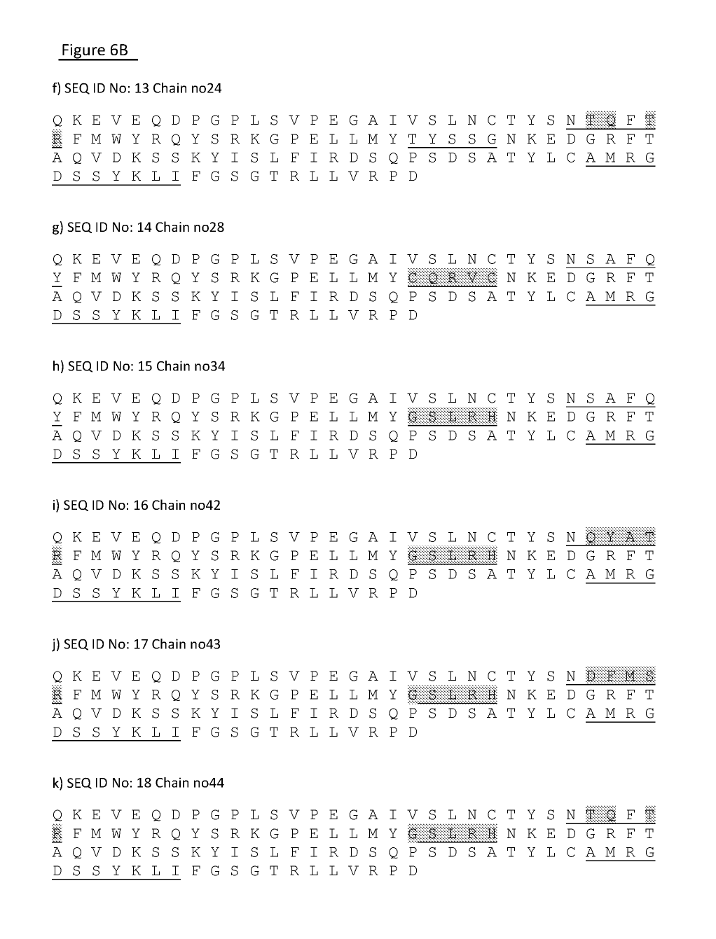

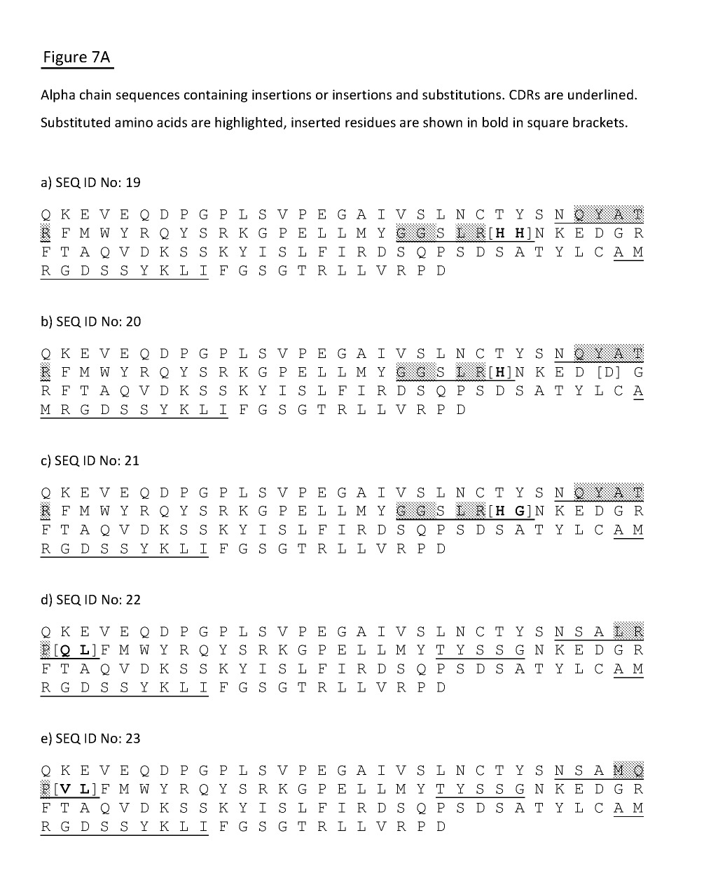

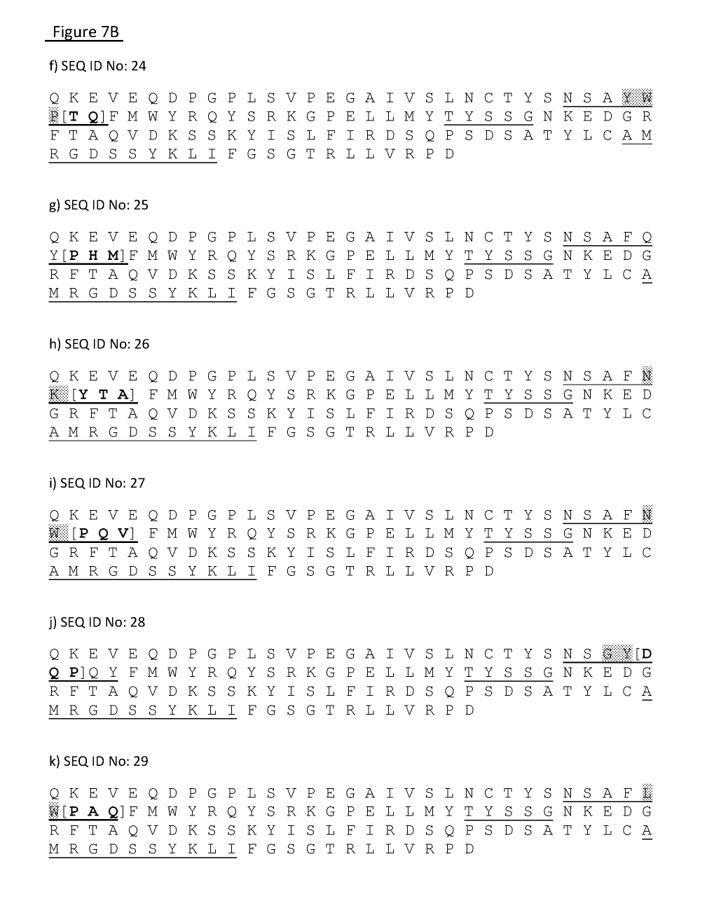

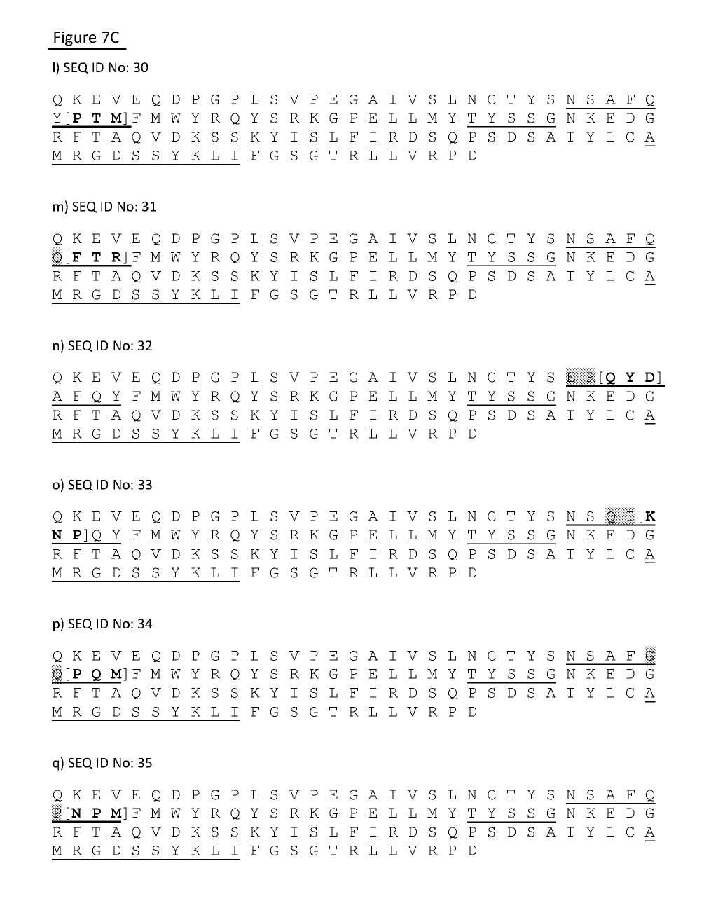

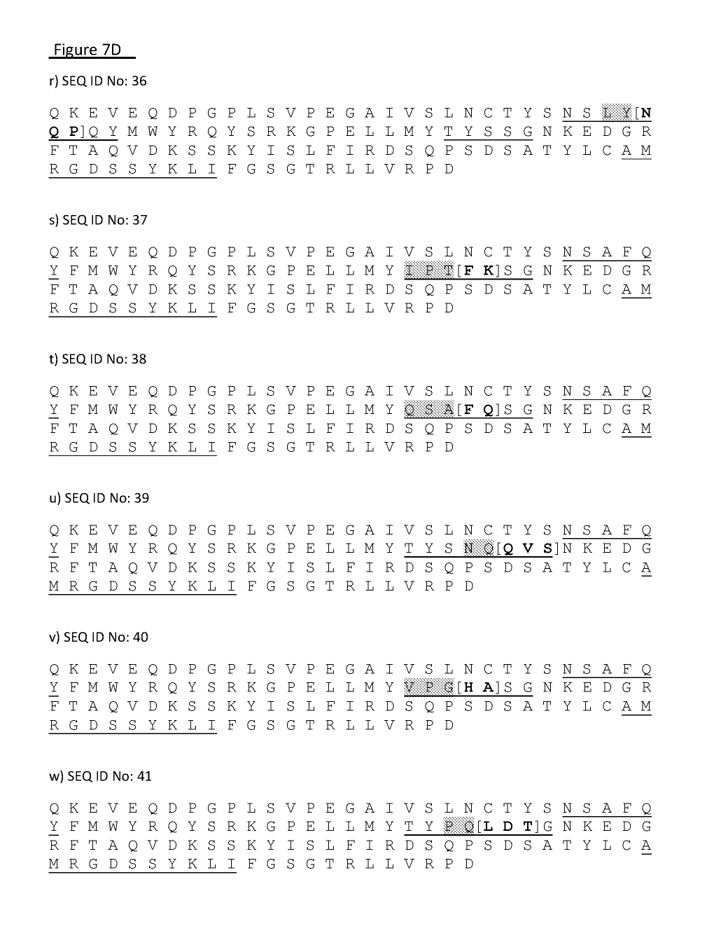

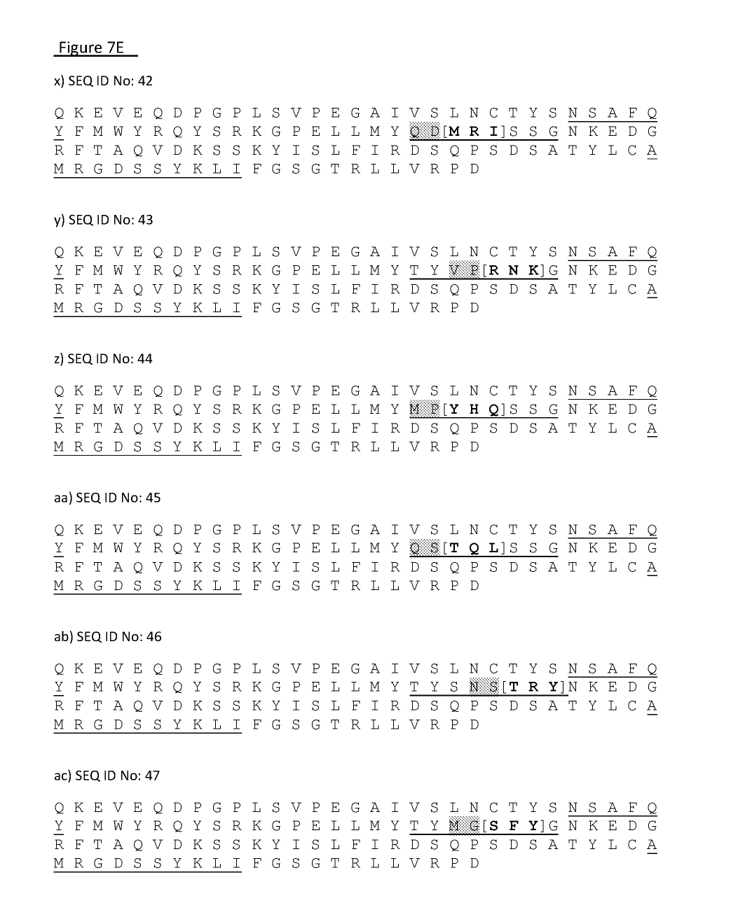

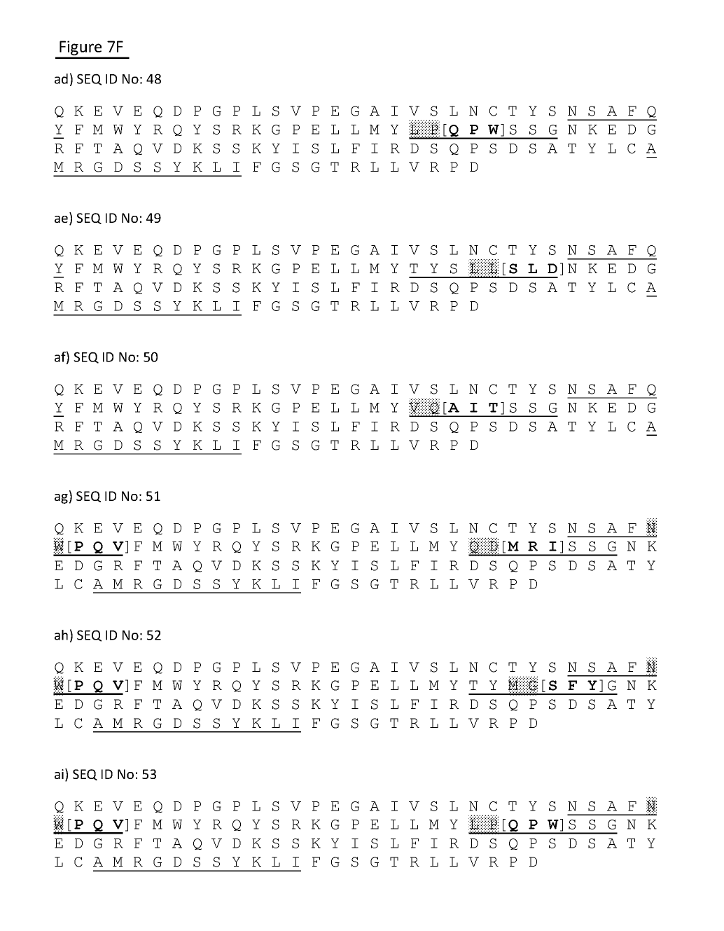

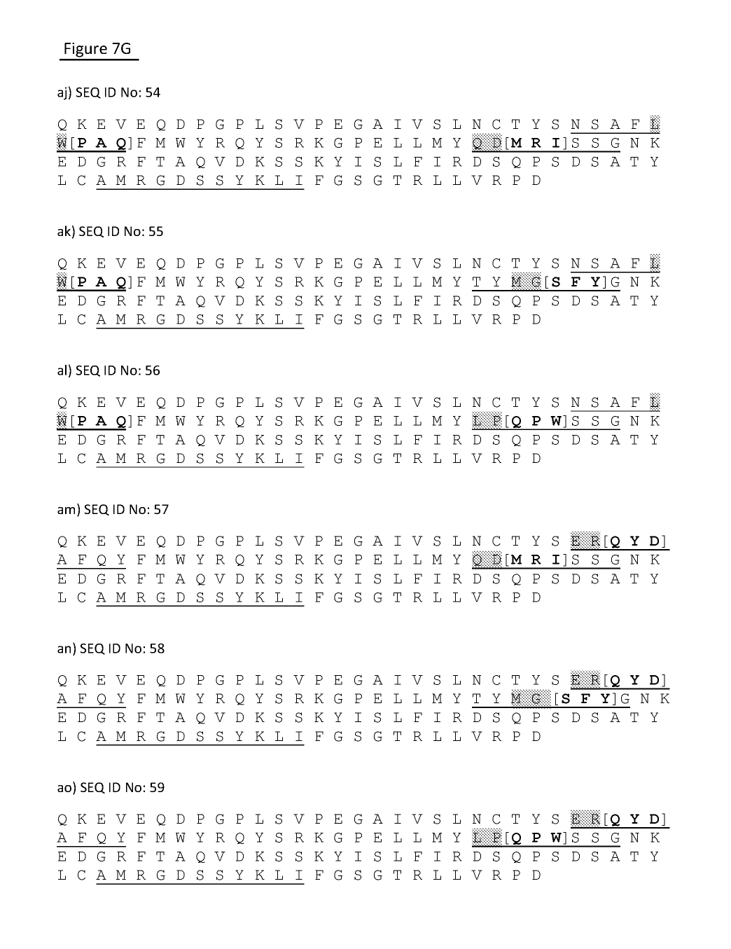

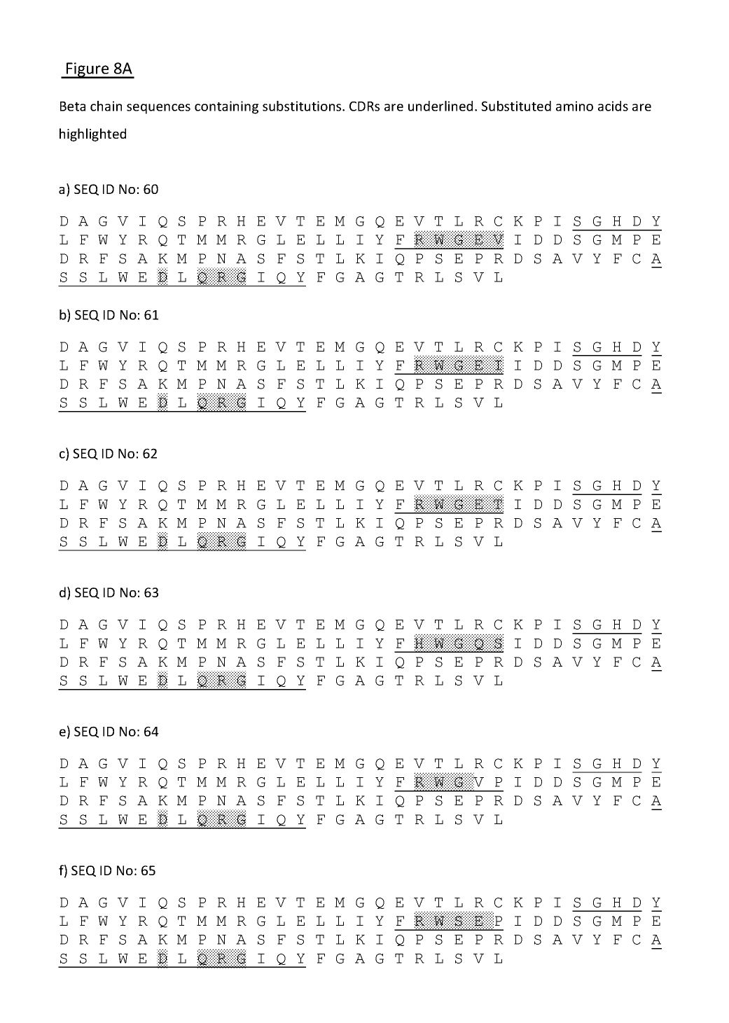

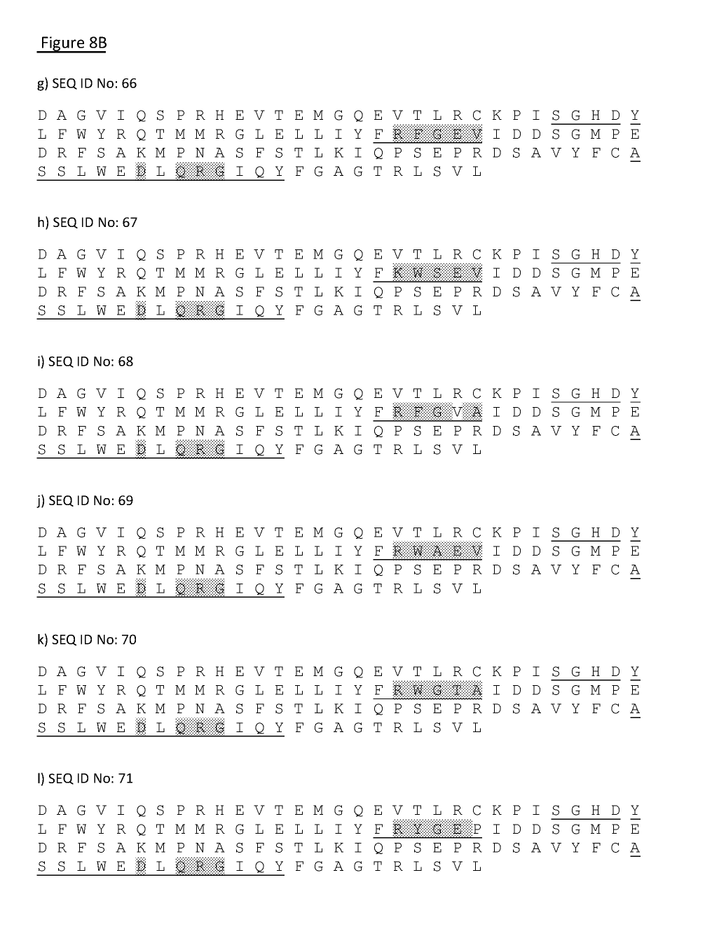

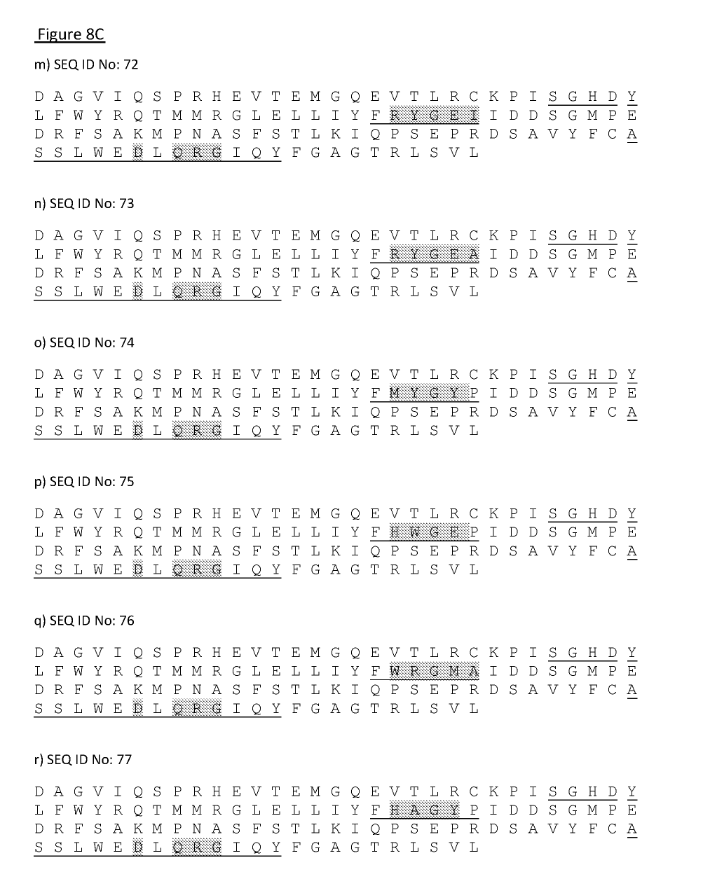

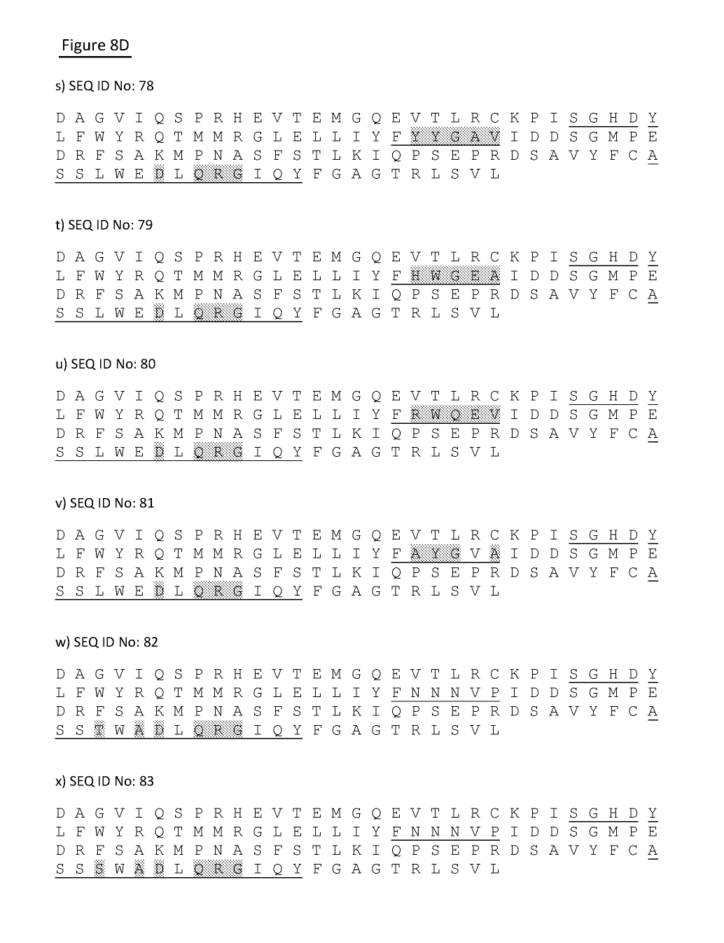

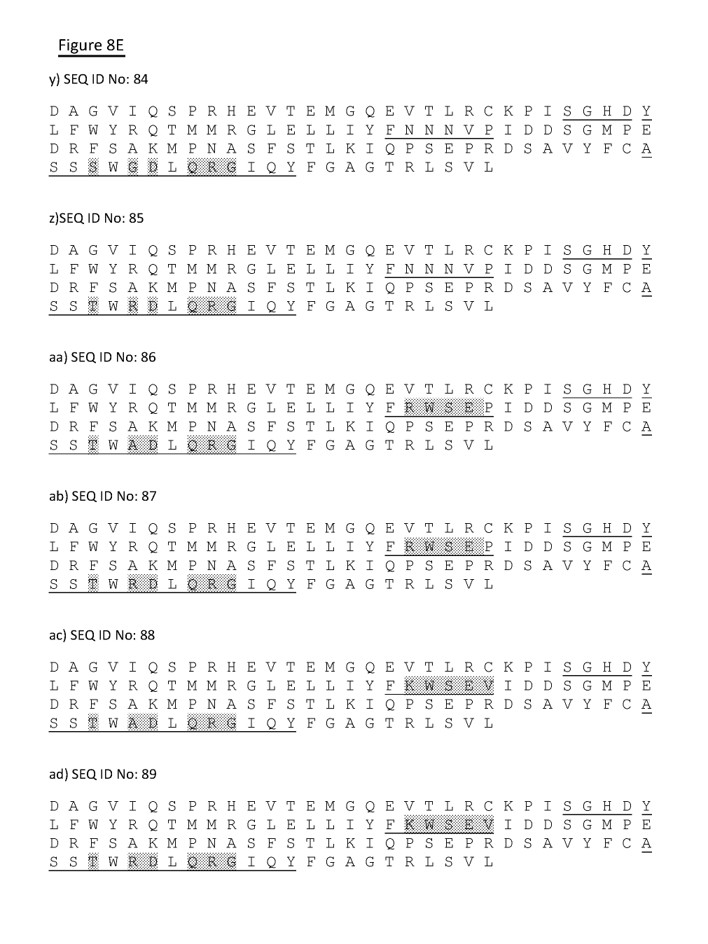

Amino acid sequences of TCR alpha and beta chain variable domains which, when combined, demonstrate improved affinity for the ALWGPDPAAA HLA-A*02 complex, compared to the reference TCR, are listed in FIGS. 6, 7 (alpha chains) and 8 (beta chains) (SEQ ID Nos: 8-59 (alpha chains) and 60-91 (beta chains))

Examples of TCR α and β chain combinations which result in improved affinity relative to the reference WT PPI TCR are as follows:—

| Alpha chain variable | Beta chain variable region | |
|---|---|---|
| region residues 1-112 | residues 1-116 (SEQ ID | |
| (SEQ ID No) | No) | K_{D} (M) |
| 2 | 60 | 2.0 × 10⁻⁶ |
| 2 | 61 | 2.0 × 10⁻⁶ |
| 2 | 62 | 3.0 × 10⁻⁶ |
| 2 | 63 | 1.32 × 10⁻⁵ |
| 2 | 64 | 1.01 × 10⁻⁵ |
| 2 | 65 | 5.7 × 10⁻⁷ |
| 2 | 66 | 1.7 × 10⁻⁶ |
| 2 | 67 | 5.6 × 10⁻⁷ |
| 2 | 68 | 1.45 × 10⁻⁶ |
| 2 | 69 | 5.0 × 10⁻⁷ |
| 2 | 70 | 1.03 × 10⁻⁶ |
| 2 | 71 | 7.8 × 10⁻⁷ |
| 2 | 72 | 6.1 × 10⁻⁷ |
| 2 | 73 | 4.0 × 10⁻⁷ |
| 2 | 74 | 7.6 × 10⁻⁸ |
| 2 | 75 | 1.19 × 10⁻⁶ |
| 2 | 76 | 2.855 × 10⁻⁵ |
| 2 | 77 | 2.388 × 10⁻⁵ |
| 2 | 78 | 1.35 × 10⁻⁵ |
| 2 | 79 | 1.7 × 10⁻⁶ |
| 2 | 80 | 8.7 × 10⁻⁷ |
| 2 | 81 | 1.42 × 10⁻⁵ |
| 2 | 82 | 2.51 × 10⁻⁵ |
| 2 | 83 | 3.96 × 10⁻⁵ |
| 2 | 84 | 6.21 × 10⁻⁵ |
| 2 | 85 | 2.89 × 10⁻⁵ |
| 2 | 86 | 2.0 × 10⁻⁷ |
| 2 | 87 | 2.2 × 10⁻⁷ |
| 2 | 88 | 1.2 × 10⁻⁷ |
| 2 | 89 | 1.5 × 10⁻⁷ |
| 2 | 90 | 1.8 × 10⁻⁸ |
| 2 | 91 | 2.9 × 10⁻⁸ |
| 8 | 74 | 1.36 × 10⁻⁸ |
| 8 | 90 | 6.26 × 10⁻⁹ |
| 9 | 74 | 3.09 × 10⁻⁸ |
| 10 | 74 | 1.76 × 10⁻⁸ |
| 10 | 90 | 5.98 × 10⁻⁹ |
| 11 | 74 | 5.81 × 10⁻⁸ |
| 12 | 74 | 4.15 × 10⁻⁸ |
| 13 | 74 | 2.59 × 10⁻⁸ |
| 13 | 90 | 3.16 × 10⁻⁹ |
| 14 | 74 | 1.37 × 10⁻⁷ |
| 15 | 74 | 7.33 × 10⁻⁸ |
| 15 | 90 | 8.27 × 10⁻⁹ |
| 16 | 90 | 3.21 × 10⁻⁹ |
| 17 | 90 | 3.4 × 10⁻⁹ |
| 18 | 90 | 6.33 × 10⁻⁹ |
| 19 | 90 | 7.42 × 10⁻⁹ |
| 20 | 90 | 8.23 × 10⁻⁹ |
| 21 | 90 | 3.91 × 10⁻⁹ |
| 22 | 90 | 4.32 × 10⁻⁸ |
| 23 | 90 | 7.47 × 10⁻⁹ |
| 24 | 90 | 5.12 × 10⁻⁹ |
| 25 | 90 | 3.21 × 10⁻⁹ |
| 26 | 90 | 5.34 × 10⁻⁹ |
| 27 | 90 | 2.1 × 10⁻⁹ |
| 28 | 90 | 1.55 × 10⁻⁹ |
| 29 | 90 | 3.35 × 10⁻⁹ |
| 30 | 90 | 3.0 × 10⁻⁸ |
| 31 | 90 | 1.38 × 10⁻⁸ |
| 32 | 90 | 6.6 × 10⁻¹⁰ |
| 33 | 90 | 5.19 × 10⁻⁸ |
| 34 | 90 | 1.16 × 10⁻⁸ |
| 35 | 90 | 1.08 × 10⁻⁸ |
| 36 | 90 | 2.95 × 10⁻⁹ |
| 37 | 90 | 1.27 × 10⁻⁸ |
| 38 | 90 | 2.21 × 10⁻⁸ |
| 39 | 90 | 3.26 × 10⁻⁸ |
| 40 | 90 | 1.62 × 10⁻⁸ |
| 41 | 90 | 8.6 × 10⁻⁹ |
| 42 | 90 | 7.1 × 10⁻⁹ |
| 43 | 90 | 7.6 × 10⁻⁹ |
| 44 | 90 | 2.18 × 10⁻⁸ |
| 45 | 90 | 2.79 × 10⁻⁸ |
| 46 | 90 | 1.66 × 10⁻⁸ |
| 47 | 90 | 8.5 × 10⁻⁹ |
| 48 | 90 | 4.6 × 10⁻⁹ |
| 49 | 90 | 5.94 × 10⁻⁸ |
| 50 | 90 | 4.08 × 10⁻⁸ |
| 51 | 90 | 8.8 × 10⁻¹⁰ |
| 52 | 90 | 8.8 × 10⁻¹⁰ |
| 53 | 90 | 9.96 × 10⁻⁹ |
| 54 | 90 | 2.17 × 10⁻⁹ |
| 55 | 90 | 3.42 × 10⁻¹⁰ |
| 56 | 90 | 1.37 × 10⁻¹⁰ |
| 57 | 90 | 3.52 × 10⁻¹⁰ |
| 58 | 90 | 2.4 × 10⁻¹⁰ |
| 59 | 90 | 2.4 × 10⁻¹⁰ |

### Example 5

### Tregs Transduced with an Affinity Enhanced TCR Prevent the Onset of Diabetes in a Mouse Model

CD25 depleted CD4+ T cells were isolated from the spleens of non-obese diabetic (NOD) mice and induced to express Foxp3 to produce a Treg phenotype. The cells were transduced with an affinity enhanced TCR (Kd=0.74 μM) specific for a peptide derived from mouse insulin. Activated cells were then injected into recipient NOD mice (n=4). A control group receiving no CD4 cells was prepared in parallel (n=3).

To induce the rapid onset of diabetes the NOD mice were injected 2 days later with an activated CD8+ T cell clone isolated from the G9 transgenic mouse (Wong et al 2009 Diabetes 58(5): 1156-1164).

Mice were monitored for urine glucose for 30 days post injection of G9 cells. Once positive, it was followed up by blood glucose tests to confirm the onset of diabetes.

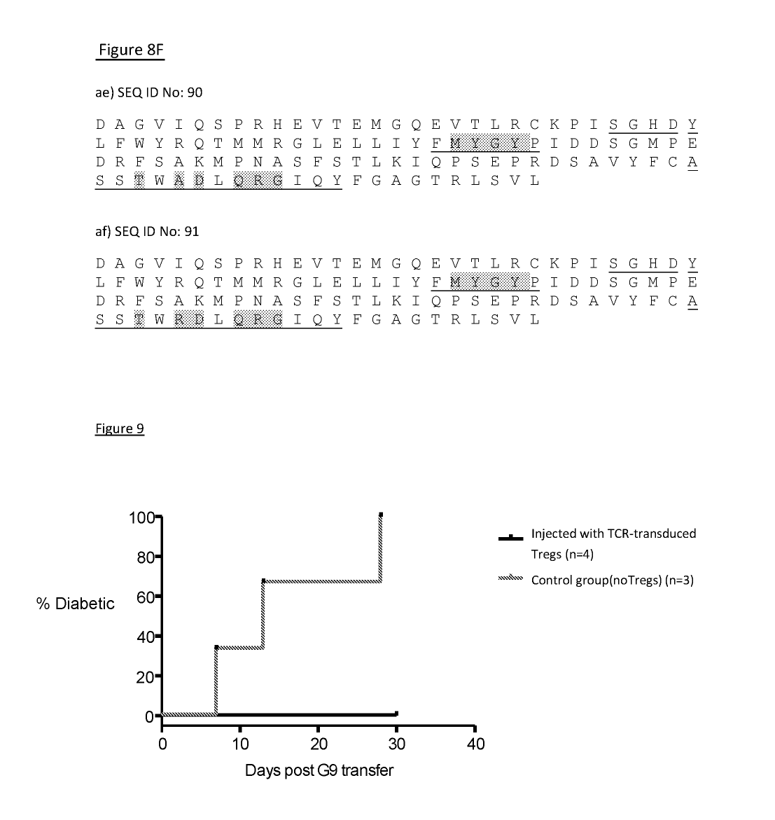

The results (FIG. 9) show that injection of TCR-transduced Tregs, prevented the onset of diabetes within the monitoring period. In contrast, mice that did not receive these cells were all positive for diabetes at the end of the 30 days.

The invention is further described by the following numbered paragraphs:
1. A T cell receptor (TCR) having the property of binding to ALWGPDPAAA (SEQ ID NO: 1) HLA-A*02 complex and comprising a TCR alpha chain variable domain and a TCR beta chain variable domain,
the alpha chain variable domain comprising an amino acid sequence that has at least 90% identity to the sequence of amino acid residues 1-112 of SEQ ID No: 2, and
the beta chain variable domain comprising an amino acid sequence that has at least 90% identity to the sequence of amino acid residues 1-116 of SEQ ID No: 3,
wherein the alpha chain variable domain has at least one of the following substitutions, with reference to the numbering of SEQ ID NO: 2:—

| Residue number | Substitutions | | | | | | | | |
|---|---|---|---|---|---|---|---|---|---|
| N27 | E | | | | | | | | |
| S28 | Q | P | D | Y | T | R | | | |
| A29 | Y | F | Q | L | G | | | | |
| F30 | A | M | Y | L | I | | | | |
| Q31 | T | S | K | R | Q | W | N | L | G |
| Y32 | R | A | V | P | K | W | Q | | |
| T50 | C | G | I | Q | V | M | L | | |
| Y51 | Q | S | G | P | D | | | | |
| S52 | R | L | S | T | A | G | V | M | |
| S53 | V | R | L | N | Q | P | G | | |
| G54 | C | H | R | Q | S | L | | | |

and/or at least one amino acid insertion,
and/or the beta chain variable domain has at least one of the following substitutions, with reference to the numbering of SEQ ID No: 3:—

| Residue number | Substitutions | | | | | | |
|---|---|---|---|---|---|---|---|
| N50 | R | H | K | M | W | Y | A |
| N51 | W | F | Y | R | A | | |
| N52 | G | S | A | Q | | | |
| V53 | E | Q | T | Y | M | A | |
| P54 | V | I | T | S | A | | |
| L96 | T | S | | | | | |
| E98 | A | G | R | | | | |
| K99 | D | | | | | | |
| A101 | Q | | | | | | |
| K102 | R | | | | | | |
| N103 | G | | | | | | |

2. The TCR of paragraph 1, wherein the alpha chain variable domain has at least one amino acid inserted immediately after the residue corresponding to S28, F30, Y32, Y51, S52, S53, G54 and/or D58, with reference to the numbering of SEQ ID NO: 2.
3. The TCR of paragraph 2, wherein the insertion is one or more of the following, with reference to the numbering of SEQ ID NO: 2:

| Residue | | | | | | | | | | | |
|---|---|---|---|---|---|---|---|---|---|---|---|
| number | Inserted residues | | | | | | | | | | |
| S28 | QYD | | | | | | | | | | |
| F30 | DQP | KNP | NQP | | | | | | | | |
| Y32 | PAQ | QL | VL | TQ | PHM | YTA | PQV | PTM | FTR | PQM | NPM |
| Y51 | QPW | MM | YHQ | TQL | AIT | | | | | | |
| S52 | FK | FQ | HA | | | | | | | | |
| S53 | SFY | LDT | RKN | | | | | | | | |
| G54 | HH | H | HG | TRY | SLD | | | | | | |
| D58 | D | | | | | | | | | | |

4. The TCR of paragraph 2 or paragraph 3, wherein the alpha chain variable domain has at least one amino acid inserted immediately after the residue corresponding to S28, Y32, Y51 and/or S53, with reference to the numbering of SEQ ID NO: 2.
5. The TCR of paragraph 4, wherein the insertion is one or more of the following, with reference to the numbering of SEQ ID NO: 2:

| Residue number | Insertion |
|---|---|
| S28 | QYD |
| Y32 | PAQ |
| Y51 | QPW or MRI |
| S53 | SFY |

6. The TCR of paragraph 4 or paragraph 5, wherein the alpha chain variable domain has (a) an insertion at S28 alone or in combination with an insertion at Y51 or S53 or (b) an insertion at Y32 alone or in combination with an insertion at Y51 or S53.
7. The TCR of any preceding paragraph, wherein the alpha chain variable domain has at least one of the following substitutions, with reference to the numbering of SEQ ID NO: 2:

| Residue number | Substitution |
|---|---|
| N27 | E |
| S28 | R |
| Q31 | L |
| Y32 | W |
| T50 | L or Q |
| Y51 | P or D |
| S52 | M |
| S53 | G |

and/or the beta chain variable domain has at least one of the following substitutions, with reference to the numbering of SEQ ID No: 3:—

| Residue number | Substitution |
|---|---|
| N50 | M |
| N51 | Y |
| N52 | G |
| V53 | Y |
| L96 | T |
| E98 | A |
| K99 | D |
| A101 | Q |
| K102 | R |
| N103 | G |

8. The TCR of paragraph 7, wherein the alpha chain variable domain has at least one of the following substitutions, with reference to the numbering of SEQ ID NO: 2:

| Residue number | Substitutions |
|---|---|
| N27 | E |
| S28 | R |
| S52 | M |
| S53 | G |

9. The TCR of any preceding paragraph, wherein the alpha chain variable domain comprises the amino acid sequence of any one of SEQ ID NOs: 8-59.
10. The TCR of any preceding paragraph, wherein the beta chain variable domain comprises the amino acid sequence of SEQ ID NOs: 60-91.
11. The TCR of any preceding paragraph having an alpha chain TRAC constant domain sequence and/or a beta chain TRBC1 or TRBC2 constant domain sequence.
12. The TCR of paragraph 11, wherein the alpha and beta chain constant domain sequences are modified by truncation or substitution to delete the native disulphide bond between Cys4 of exon 2 of TRAC and Cys2 of exon 2 of TRBC1 or TRBC2.
13. The TCR of paragraph 11 or paragraph 13, wherein the alpha and beta chain constant domain sequences are modified by substitution of cysteine residues for Thr 48 of TRAC and Ser 57 of TRBC1 or TRBC2, the cysteines forming a disulphide bond between the alpha and beta constant domains of the TCR.
14. The TCR of any preceding paragraph, which is in single chain format of the type: Vα-L-Vβ, Vα-Cα-L-Vβ, Vα-L-Vβ-Cβ or Vα-Cα-L-Vβ-Cβ, optionally in the reverse orientation, wherein Vα and Vβ represent TCR α and β variable regions respectively, Cα and Cβ represent TCR α and β constant regions respectively, and L represents a linker sequence.
15. The TCR of any one of paragraphs 1 to 13, which is an alpha-beta heterodimer.
16. The TCR of any preceding paragraph associated with a detectable label, a therapeutic agent or a PK modifying moiety.
17. A nucleic acid comprising a sequence encoding an α chain variable domain of a TCR as in any preceding paragraph and/or a sequence encoding a β chain variable domain of a TCR as in any preceding paragraph.
18. A non-naturally occurring and/or purified and/or engineered cell, preferably a T-cell, more preferably a Treg cell presenting a TCR as in any one of paragraphs 1 to 19.
19. A pharmaceutical composition comprising a TCR as in any one of paragraphs 1 to 16, a nucleic acid as in paragraph 17 and/or a cell as in paragraph 18, together with one or more pharmaceutically acceptable carriers or excipients.
20. A TCR T cell receptor (TCR) having the property of binding to ALWGPDPAAA (SEQ ID No: 1) HLA-A*02 complex, or a cell expressing and/or presenting such a TCR, for use in medicine.
21. The TCR or cell for use of paragraph 20, for use in a method of treating type I diabetes.
22. The TCR or cell for use of paragraph 21, wherein the method comprises adoptive therapy.
23. The TCR or cell for use of any one of paragraph 20 to 22, wherein the TCR is in any one of paragraphs 1 to 16 and/or wherein the cell is in paragraph 17.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A T cell receptor (TCR) having the property of binding to ALWGPDPAAA (SEQ ID NO: 1) HLA-A*02 complex and comprising a TCR alpha chain variable domain and a TCR beta chain variable domain,
the alpha chain variable domain comprising an amino acid sequence that has at least 90% identity to the sequence of amino acid residues 1-112 of SEQ ID No: 2, and
the beta chain variable domain comprising an amino acid sequence that has at least 90% identity to the sequence of amino acid residues 1-116 of SEQ ID No: 3,
wherein the alpha chain variable domain has at least one of the following substitutions, with reference to the numbering of SEQ ID NO: 2:—

| Residue number | Substitutions | | | | | | | | |
|---|---|---|---|---|---|---|---|---|---|
| N27 | E | | | | | | | | |
| S28 | Q | P | D | Y | T | R | | | |
| A29 | Y | F | Q | L | G | | | | |
| F30 | A | M | Y | L | I | | | | |
| Q31 | T | S | K | R | Q | W | N | L | G |
| Y32 | R | A | V | P | K | W | Q | | |
| T50 | C | G | I | Q | V | M | L | | |
| Y51 | Q | S | G | P | D | | | | |
| S52 | R | L | S | T | A | G | V | M | |
| S53 | V | R | L | N | Q | P | G | | |
| G54 | C | H | R | Q | S | L | | | |

and/or at least one amino acid insertion,
and/or the beta chain variable domain has at least one of the following substitutions, with reference to the numbering of SEQ ID No: 3:—

| Residue number | Substitutions | | | | | | |
|---|---|---|---|---|---|---|---|
| N50 | R | H | K | M | W | Y | A |
| N51 | W | F | Y | R | A | | |
| N52 | G | S | A | Q | | | |
| V53 | E | Q | T | Y | M | A | |
| P54 | V | I | T | S | A | | |
| L96 | T | S | | | | | |
| E98 | A | G | R | | | | |
| K99 | D | | | | | | |
| A101 | Q | | | | | | |
| K102 | R | | | | | | |
| N103 | G | | | | | | |

2. The TCR of claim 1, wherein the alpha chain variable domain has at least one amino acid inserted immediately after the residue corresponding to S28, F30, Y32, Y51, S52, S53, G54 and/or D58, with reference to the numbering of SEQ ID NO: 2.

3. The TCR of claim 2, wherein the insertion is one or more of the following, with reference to the numbering of SEQ ID NO: 2:

| Residue | | | | | | | | | | | |
|---|---|---|---|---|---|---|---|---|---|---|---|
| number | Inserted residues | | | | | | | | | | |
| S28 | QYD | | | | | | | | | | |
| F30 | DQP | KNP | NQP | | | | | | | | |
| Y32 | PAQ | QL | VL | TQ | PHM | YTA | PQV | PTM | FTR | PQM | NPM |
| Y51 | QPW | MM | YHQ | TQL | AIT | | | | | | |
| S52 | FK | FQ | HA | | | | | | | | |
| S53 | SFY | LDT | RKN | | | | | | | | |
| G54 | HH | H | HG | TRY | SLD | | | | | | |
| D58 | D | | | | | | | | | | |

4. The TCR of claim 2, wherein the alpha chain variable domain has at least one amino acid inserted immediately after the residue corresponding to S28, Y32, Y51 and/or S53, with reference to the numbering of SEQ ID NO: 2.

5. The TCR of claim 4, wherein the insertion is one or more of the following, with reference to the numbering of SEQ ID NO: 2:

| Residue number | Insertion |
|---|---|
| S28 | QYD |
| Y32 | PAQ |
| Y51 | QPW or MRI |
| S53 | SFY |

6. The TCR of claim 4, wherein the alpha chain variable domain has (a) an insertion at S28 alone or in combination with an insertion at Y51 or S53 or (b) an insertion at Y32 alone or in combination with an insertion at Y51 or S53.

7. The TCR of claim 1, wherein the alpha chain variable domain has at least one of the following substitutions, with reference to the numbering of SEQ ID NO: 2:

| Residue number | Substitution |
|---|---|
| N27 | E |
| S28 | R |
| Q31 | L |
| Y32 | W |
| T50 | L or Q |
| Y51 | P or D |
| S52 | M |
| S53 | G |

and/or the beta chain variable domain has at least one of the following substitutions, with reference to the numbering of SEQ ID No: 3:—

| Residue number | Substitution |
|---|---|
| N50 | M |
| N51 | Y |
| N52 | G |
| V53 | Y |
| L96 | T |
| E98 | A |
| K99 | D |
| A101 | Q |
| K102 | R |
| N103 | G |

8. The TCR of claim 7, wherein the alpha chain variable domain has at least one of the following substitutions, with reference to the numbering of SEQ ID NO: 2:

| Residue number | Substitutions |
|---|---|
| N27 | E |
| S28 | R |
| S52 | M |
| S53 | G |

9. The TCR of claim 1, wherein the alpha chain variable domain comprises the amino acid sequence of any one of SEQ ID NOs: 8-59.

10. The TCR of claim 1, wherein the beta chain variable domain comprises the amino acid sequence of SEQ ID NOs: 60-91.

11. The TCR of claim 1 having an alpha chain TRAC constant domain sequence and/or a beta chain TRBC1 or TRBC2 constant domain sequence.

12. The TCR of claim 11, wherein the alpha and beta chain constant domain sequences are modified by truncation or substitution to delete the native disulphide bond between Cys4 of exon 2 of TRAC and Cys2 of exon 2 of TRBC1 or TRBC2.

13. The TCR of claim 11, wherein the alpha and beta chain constant domain sequences are modified by substitution of cysteine residues for Thr 48 of TRAC and Ser 57 of TRBC1 or TRBC2, the cysteines forming a disulphide bond between the alpha and beta constant domains of the TCR.

14. The TCR of claim 1, which is in single chain format of the type: Vα-L-Vβ, Vα-Cα-L-Vβ, Vα-L-Vβ-Cβ or Vα-Cα-L-Vβ-Cβ, optionally in the reverse orientation, wherein Vα and VP represent TCR α and β variable regions respectively, Cα and Cβ represent TCR α and β constant regions respectively, and L represents a linker sequence.

15. The TCR of claim 1, which is an alpha-beta heterodimer.

16. The TCR of claim 1 associated with a detectable label, a therapeutic agent or a PK modifying moiety.

17. A nucleic acid comprising a sequence encoding an α chain variable domain of a TCR as claimed in claim 1 and/or a sequence encoding a β chain variable domain of a TCR as claimed in claim 1.

18. A non-naturally occurring and/or purified and/or engineered cell, preferably a T-cell, more preferably a Treg cell presenting a TCR as claimed in claim 1.

19. A pharmaceutical composition comprising a TCR as claimed in claim 1, together with one or more pharmaceutically acceptable carriers or excipients.

20. A pharmaceutical composition comprising a nucleic acid as claimed in claim 17, together with one or more pharmaceutically acceptable carriers or excipients.

21. A pharmaceutical composition comprising a cell as claimed in claim 18, together with one or more pharmaceutically acceptable carriers or excipients.
